SEQUENCE LISTING (27 sequences)

SEQ ID NO: 1 - protein, 477 aa, Homo sapiens
MKSLPILLLLCVAVCSAYPLDGAARGEDTSMNLVQKYLENYYDLKKDVKQFVRRKDSGPVVKKIREMQKFLGLEVTGKLDSDTLEVMRKPRCGVPDVGHFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGPPPDSPETPLVPTEPVPPEPGTPANCDPALSFDAVSTLRGEILIFKDRHFWRKSLRKLEPELHLISSFWPSLPSGVDAAYEVTSKDLVFIFKGNQFWAIRGNEVRAGYPRGIHTLGFPPTVRKIDAAISDKEKNKTYFFVEDKYWRFDEKRNSMEPGFPKQIAEDFPGIDSKIDAVFEEFGFFYFFTGSSQLEFDPNAKKVTHTLKSNSWLNC

SEQ ID NO: 2 - protein, 377 aa, Artificial Sequence
  Recombinant human MMP-3 amino acid sequence (lacking pro-peptide domain)
RTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGPPPDSPETPLVPTEPVPPEPGTPANCDPALSFDAVSTLRGEILIFKDRHFWRKSLRKLEPELHLISSFWPSLPSGVDAAYEVTSKDLVFIFKGNQFWAIRGNEVRAGYPRGIHTLGFPPTVRKIDAAISDKEKNKTYFFVEDKYWRFDEKRNSMEPGFPKQIAEDFPGIDSKIDAVFEEFGFFYFFTGSSQLEFDPNAKKVTHTLKSNSWLNC

SEQ ID NO: 3 - DNA, 3503 bp, Artificial Sequence
  Full length vector (not including backbone)
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatccatgaagagtcttccaatcctactgttgctgtgcgtggcagtttgctcagcctatccattggatggagctgcaaggggtgaggacaccagcatgaaccttgttcagaaatatctagaaaactactacgacctcaaaaaagatgtgaaacagtttgttaggagaaaggacagtggtcctgttgttaaaaaaatccgagaaatgcagaagttccttggattggaggtgacggggaagctggactccgacactctggaggtgatgcgcaagcccaggtgtggagttcctgatgttggtcacttcagaacctttcctggcatcccgaagtggaggaaaacccaccttacatacaggattgtgaattatacaccagatttgccaaaagatgctgttgattctgctgttgagaaagctctgaaagtctgggaagaggtgactccactcacattctccaggctgtatgaaggagaggctgatataatgatctcttttgcagttagagaacatggagacttttacccttttgatggacctggaaatgttttggcccatgcctatgcccctgggccagggattaatggagatgcccactttgatgatgatgaacaatggacaaaggatacaacagggaccaatttatttctcgttgctgctcatgaaattggccactccctgggtctctttcactcagccaacactgaagctttgatgtacccactctatcactcactcacagacctgactcggttccgcctgtctcaagatgatataaatggcattcagtccctctatggacctccccctgactcccctgagacccccctggtacccacggaacctgtccctccagaacctgggacgccagccaactgtgatcctgctttgtcctttgatgctgtcagcactctgaggggagaaatcctgatctttaaagacaggcacttttggcgcaaatccctcaggaagcttgaacctgaattgcatttgatctcttcattttggccatctcttccttcaggcgtggatgccgcatatgaagttactagcaaggacctcgttttcatttttaaaggaaatcaattctgggctatcagaggaaatgaggtacgagctggatacccaagaggcatccacaccctaggtttccctccaaccgtgaggaaaatcgatgcagccatttctgataaggaaaagaacaaaacatatttctttgtagaggacaaatactggagatttgatgagaagagaaattccatggagccaggctttcccaagcaaatagctgaagactttccagggattgactcaaagattgatgctgtttttgaagaatttgggttcttttatttctttactggatcttcacagttggagtttgacccaaatgcaaagaaagtgacacacactttgaagagtaacagctggcttaattgttgatacccatacgatgttccagattacgctgacccacatattgaagagcaatagctggtttaattgttaactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 4 - DNA, 3232 bp, Artificial Sequence
  Expression cassette (not including ITRs)
gcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatccatgaagagtcttccaatcctactgttgctgtgcgtggcagtttgctcagcctatccattggatggagctgcaaggggtgaggacaccagcatgaaccttgttcagaaatatctagaaaactactacgacctcaaaaaagatgtgaaacagtttgttaggagaaaggacagtggtcctgttgttaaaaaaatccgagaaatgcagaagttccttggattggaggtgacggggaagctggactccgacactctggaggtgatgcgcaagcccaggtgtggagttcctgatgttggtcacttcagaacctttcctggcatcccgaagtggaggaaaacccaccttacatacaggattgtgaattatacaccagatttgccaaaagatgctgttgattctgctgttgagaaagctctgaaagtctgggaagaggtgactccactcacattctccaggctgtatgaaggagaggctgatataatgatctcttttgcagttagagaacatggagacttttacccttttgatggacctggaaatgttttggcccatgcctatgcccctgggccagggattaatggagatgcccactttgatgatgatgaacaatggacaaaggatacaacagggaccaatttatttctcgttgctgctcatgaaattggccactccctgggtctctttcactcagccaacactgaagctttgatgtacccactctatcactcactcacagacctgactcggttccgcctgtctcaagatgatataaatggcattcagtccctctatggacctccccctgactcccctgagacccccctggtacccacggaacctgtccctccagaacctgggacgccagccaactgtgatcctgctttgtcctttgatgctgtcagcactctgaggggagaaatcctgatctttaaagacaggcacttttggcgcaaatccctcaggaagcttgaacctgaattgcatttgatctcttcattttggccatctcttccttcaggcgtggatgccgcatatgaagttactagcaaggacctcgttttcatttttaaaggaaatcaattctgggctatcagaggaaatgaggtacgagctggatacccaagaggcatccacaccctaggtttccctccaaccgtgaggaaaatcgatgcagccatttctgataaggaaaagaacaaaacatatttctttgtagaggacaaatactggagatttgatgagaagagaaattccatggagccaggctttcccaagcaaatagctgaagactttccagggattgactcaaagattgatgctgtttttgaagaatttgggttcttttatttctttactggatcttcacagttggagtttgacccaaatgcaaagaaagtgacacacactttgaagagtaacagctggcttaattgttgatacccatacgatgttccagattacgctgacccacatattgaagagcaatagctggtttaattgttaactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 5 - DNA, 130 bp, Artificial Sequence
  AAV2 ITR  1-130  130bp
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 6 - DNA, 304 bp, Artificial Sequence
  CMV enhancer 210-513  304bp
cgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 7 - DNA, 203 bp, Artificial Sequence
  CMV promoter 514-716  203bp
gtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagct

SEQ ID NO: 8 - DNA, 493 bp, Artificial Sequence
  Human beta-globin Intron  809-1301  493bp
cgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggat

SEQ ID NO: 9 - DNA, 1434 bp, Artificial Sequence
  Human MMP3  1332-2765  1434bp
atgaagagtcttccaatcctactgttgctgtgcgtggcagtttgctcagcctatccattggatggagctgcaaggggtgaggacaccagcatgaaccttgttcagaaatatctagaaaactactacgacctcaaaaaagatgtgaaacagtttgttaggagaaaggacagtggtcctgttgttaaaaaaatccgagaaatgcagaagttccttggattggaggtgacggggaagctggactccgacactctggaggtgatgcgcaagcccaggtgtggagttcctgatgttggtcacttcagaacctttcctggcatcccgaagtggaggaaaacccaccttacatacaggattgtgaattatacaccagatttgccaaaagatgctgttgattctgctgttgagaaagctctgaaagtctgggaagaggtgactccactcacattctccaggctgtatgaaggagaggctgatataatgatctcttttgcagttagagaacatggagacttttacccttttgatggacctggaaatgttttggcccatgcctatgcccctgggccagggattaatggagatgcccactttgatgatgatgaacaatggacaaaggatacaacagggaccaatttatttctcgttgctgctcatgaaattggccactccctgggtctctttcactcagccaacactgaagctttgatgtacccactctatcactcactcacagacctgactcggttccgcctgtctcaagatgatataaatggcattcagtccctctatggacctccccctgactcccctgagacccccctggtacccacggaacctgtccctccagaacctgggacgccagccaactgtgatcctgctttgtcctttgatgctgtcagcactctgaggggagaaatcctgatctttaaagacaggcacttttggcgcaaatccctcaggaagcttgaacctgaattgcatttgatctcttcattttggccatctcttccttcaggcgtggatgccgcatatgaagttactagcaaggacctcgttttcatttttaaaggaaatcaattctgggctatcagaggaaatgaggtacgagctggatacccaagaggcatccacaccctaggtttccctccaaccgtgaggaaaatcgatgcagccatttctgataaggaaaagaacaaaacatatttctttgtagaggacaaatactggagatttgatgagaagagaaattccatggagccaggctttcccaagcaaatagctgaagactttccagggattgactcaaagattgatgctgtttttgaagaatttgggttcttttatttctttactggatcttcacagttggagtttgacccaaatgcaaagaaagtgacacacactttgaagagtaacagctggcttaattgttga

SEQ ID NO: 10 - protein, 477 aa, Artificial Sequence; identical to SEQ ID NO: 1
  Human MMP3  1332-2765  1434bp (amino acid)

SEQ ID NO: 11 - DNA, 477 bp, Artificial Sequence
  hGH poly(A) signal   2847-3323   477bp
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 12 - DNA, 141 bp, Artificial Sequence
  AAV2 ITR (inverted) 3363-3503  141bp
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 13 - protein, 736 aa, Artificial Sequence
  AAV9 capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - DNA, 119 bp, Artificial Sequence
  5′ ITR
ctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 15 - DNA, 145 bp, Artificial Sequence
  5′ ITR
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaac

SEQ ID NO: 16 - DNA, 130 bp, Artificial Sequence
  3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 17 - DNA, 145 bp, Artificial Sequence
  3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 18 - DNA, 145 bp, Artificial Sequence
  3′ ITR
gttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 19 - DNA, 663 bp, Artificial Sequence
  CMV enhancer/promoter
acgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcc

SEQ ID NO: 20 - DNA, 480 bp, Artificial Sequence
  hGH polyA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctg

SEQ ID NO: 21 - DNA, 10 bp, Artificial Sequence
  Kozak
cgccaccatg

SEQ ID NO: 22 - protein, 453 aa, Homo sapiens
RGEDTSMNLVQKYLENYYDLKKDVKQFVRRKDSGPVVKKIREMQKFLGLEVTGKLDSDTLEVMRKPRCGVPDVGHFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGPPPDSPETPLVPTEPVPPEPGTPANCDPALSFDAVSTLRGEILIFKDRHFWRKSLRKLEPELHLISSFWPSLPSGVDAAYEVTSKDLVFIFKGNQFWAIRGNEVRAGYPRGIHTLGFPPTVRKIDAAISDKEKNKTYFFVEDKYWRFDEKRNSMEPGFPKQIAEDFPGIDSKIDAVFEEFGFFYFFTGSSQLEFDPNAKKVTHTLKSNSWLNC

SEQ ID NO: 23 - DNA, 1434 bp, Artificial Sequence
  Optimized MMP3 CDNA 1
atgaagtcgctgccgattctcctgctgctgtgcgtggccgtgtgcagcgcctacccgctggacggcgccgcccggggagaggacacctccatgaacctcgtgcagaagtacctggaaaactactacgatctcaaaaaggacgtgaagcagtttgtgcggcggaaggactccggtcctgtggtcaagaagattcgggagatgcagaagttcctgggccttgaagtgaccggaaagctcgatagcgataccctcgaagtcatgcggaagccccgctgcggagtccctgacgtgggacatttcaggacttttcctggcatccctaagtggagaaagacccacctcacgtaccgcatcgtgaactatacccccgatctgcctaaggacgcagtggactccgccgtggaaaaggcactgaaagtctgggaagaagtcacaccactgaccttctcccgcctgtatgaaggggaagccgacattatgatctcgttcgctgtgcgggagcacggggatttctacccatttgacgggcccggaaacgtgctggcgcatgcctacgcgcccggcccgggaatcaacggcgatgcacatttcgacgacgacgagcagtggaccaaggacaccactggtaccaatctgttcctggtggccgcccacgagattggtcattcactgggcctgtttcactccgccaacactgaagctctgatgtacccgctgtaccactcactgactgacctgaccagattccgcttgagccaagacgacatcaacggaatccagtcactgtacggtcccccgcccgactcgcctgaaacccccttggtgcccaccgagcccgtgcccccggagccgggaactccggccaactgcgacccagccctgtccttcgacgctgtctcgactcttcgcggagagatcctgattttcaaggatcggcacttctggcgaaagtcgcttaggaaactcgagccagagctccacctgatctcctccttctggccgagcttgccctccggcgtggatgccgcttacgaagtgacctcgaaagatcttgtgttcatattcaagggaaaccagttctgggcgatcaggggaaacgaagtcagagcgggctaccctagagggatccacactctggggttccctccaaccgtgcggaagattgatgccgcgattagcgataaggaaaagaacaagacttacttcttcgtggaggacaagtactggcgcttcgacgaaaagcgcaactccatggaaccgggatttccgaagcaaatcgccgaggatttcccgggtatcgactccaagatcgacgccgtgttcgaagagttcggcttcttctacttcttcaccggctcctcccaactcgagtttgaccctaacgccaagaaggtcacccacacgctcaagtccaactcttggctgaattgttga

SEQ ID NO: 24 - DNA, 1434 bp, Artificial Sequence
  Optimized MMP3 CDNA 2
atgaagtccctcccgatcctcctgcttctgtgcgtggcagtgtgcagcgcctacccgctggatggggctgccaggggcgaagatacttcgatgaatctggtgcagaagtacctggagaactactatgacttgaagaaagacgtgaagcagttcgtgcggagaaaggactccgggccagtggtcaagaagattcgcgaaatgcagaagttcctgggcctcgaagtgaccggaaaactggactccgatactctggaagtcatgcgcaagcctagatgcggcgtgcccgacgtgggccacttccggacattccctggaatcccgaagtggagaaagacccacttgacctaccggatcgtgaactacactcccgatctgcctaaggacgccgtggactccgccgtggaaaaggccctcaaagtctgggaagaagtgaccccgctgacctttagccgcctgtacgagggcgaagcggacatcatgatttccttcgccgtgcgggagcacggggatttctaccctttcgacggaccgggaaacgtgctggcccacgcttacgcgcccggtcccggcattaacggcgatgctcacttcgacgacgatgagcagtggaccaaggataccactggaaccaacctgttccttgtggccgcccatgaaattgggcactcactgggactgtttcattccgccaacaccgaggccctgatgtacccgctgtaccactccctcaccgacttgactagattccgcctgtcacaagacgacattaacgggatccagtccctctacggtcccccgccggactcgccggaaaccccgcttgtgcctaccgaaccagtgccgcccgagcctggcactcccgccaactgcgaccctgccctgagcttcgacgcagtcagcaccctgcgcggagagatcctgatctttaaggaccgacacttctggcggaaatcactgcgcaagctcgaaccagagctccatctcatctcgtccttctggccctccctgccgtctggagtggacgcagcctacgaggtcacgtccaaggatctggtgttcatattcaagggaaaccagttctgggcgatcaggggcaacgaagtcagagcgggatacccccggggcattcacactttgggatttcctcctaccgtgcggaagattgacgccgcgatctcggacaaggaaaagaacaagacttacttcttcgtcgaggacaagtattggcggttcgatgaaaagcgcaactccatggaacccggtttcccaaagcaaattgctgaggacttccccggcatcgactcaaagatcgatgccgtgttcgaggagtttgggttcttctacttcttcaccggttcgagccagctcgagttcgatccaaacgccaagaaagtcactcacaccctgaagtccaacagctggctgaattgttaa

SEQ ID NO: 25 - DNA, 1434 bp, Artificial Sequence
  Optimized MMP3 CDNA 3
atgaagtcactgccgatcctgttgctgctgtgcgtggccgtgtgctcggcttaccccctggatggcgccgcgcgcggagaggacacctccatgaaccttgtgcaaaagtacctcgagaactactacgacctgaagaaggatgtgaagcagttcgtgcgccgcaaggattcgggacctgtggtcaaaaagatccgcgagatgcagaaattccttggactcgaagtgaccggaaagctcgactccgacactcttgaagtcatgagaaagccgcgctgcggagtcccggacgtgggccatttccggaccttccccggtattccgaagtggagaaagactcacctgacataccgcatcgtgaactacactcccgacctcccgaaggacgcagtggactcggccgtggaaaaggccttgaaagtctgggaggaagtgacccctctgaccttttcgagactgtacgaaggggaagccgatatcatgatctccttcgccgtgcgggaacacggggacttttatccttttgatggccctgggaacgtcctggcgcatgcttacgcccctggacccgggatcaacggagatgcccacttcgacgatgatgagcagtggaccaaggacaccaccggtactaacctgttcctggtggccgcccacgaaatcggtcattccctgggactcttccattccgcgaacactgaagccctgatgtacccgttgtaccactccctgaccgacctcactcggttccggctgagccaggacgatatcaacggcattcagagcctctatggacccccgcctgattcccccgaaaccccgctggtgccgaccgagccagtgccaccagagccgggaactcccgcgaactgcgaccccgctctgagctttgacgctgtgtcaaccctgaggggcgaaattctgattttcaaggacagacacttctggcgcaagagcctccggaagctcgagcctgagctgcacctcatttcctcgttctggccaagccttccgtccggagtggacgccgcatacgaagtcacgtccaaagacctggtgttcatctttaagggcaatcagttctgggcgattcggggcaacgaagtcagggccggctaccctcggggcatccacaccctggggtttccccccactgtgcggaagatcgatgcagccatcagcgacaaggaaaagaacaagacctacttcttcgtggaggacaagtactggcggttcgatgagaagcgaaatagcatggaacccggcttcccgaagcaaattgccgaggacttcccgggtatcgactccaagatcgacgccgtgttcgaggagttcggtttcttctacttcttcaccgggtcatctcagctggaattcgaccccaacgcgaagaaagtcactcacaccctcaagtccaactcctggctcaactgttga

SEQ ID NO: 26 - DNA, 1434 bp, Artificial Sequence
  Native MMP3 CpG depleted
atgaagagtcttccaatcctactgttgctgtgtgtggcagtttgctcagcctatccattggatggagctgcaaggggtgaggacaccagcatgaaccttgttcagaaatatctagaaaactactatgacctcaaaaaagatgtgaaacagtttgttaggagaaaggacagtggtcctgttgttaaaaaaatcagagaaatgcagaagttccttggattggaggtgacagggaagctggactctgacactctggaggtgatgagaaagcccaggtgtggagttcctgatgttggtcacttcagaacctttcctggcatccccaagtggaggaaaacccaccttacatacaggattgtgaattatacaccagatttgccaaaagatgctgttgattctgctgttgagaaagctctgaaagtctgggaagaggtgactccactcacattctccaggctgtatgaaggagaggctgatataatgatctcttttgcagttagagaacatggagacttttacccttttgatggacctggaaatgttttggcccatgcctatgcccctgggccagggattaatggagatgcccactttgatgatgatgaacaatggacaaaggatacaacagggaccaatttatttctggttgctgctcatgaaattggccactccctgggtctctttcactcagccaacactgaagctttgatgtacccactctatcactcactcacagacctgactagattcagactgtctcaagatgatataaatggcattcagtccctctatggacctccccctgactcccctgagacccccctggtacccacagaacctgtccctccagaacctgggaccccagccaactgtgatcctgctttgtcctttgatgctgtcagcactctgaggggagaaatcctgatctttaaagacaggcacttttggagaaaatccctcaggaagcttgaacctgaattgcatttgatctcttcattttggccatctcttccttcaggggtggatgctgcatatgaagttactagcaaggacctggttttcatttttaaaggaaatcaattctgggctatcagaggaaatgaggtaagagctggatacccaagaggcatccacaccctaggtttccctccaacagtgaggaaaattgatgcagccatttctgataaggaaaagaacaaaacatatttctttgtagaggacaaatactggagatttgatgagaagagaaattccatggagccaggctttcccaagcaaatagctgaagactttccagggattgactcaaagattgatgctgtttttgaagaatttgggttcttttatttctttactggatcttcacagttggagtttgacccaaatgcaaagaaagtgacacacactttgaagagtaacagctggcttaattgttga

SEQ ID NO: 27 - DNA, 1434 bp, Artificial Sequence
  MMP3 Opt 3 CpG depleted
atgaagtcactgcctatcctgttgctgctgtgtgtggctgtgtgctctgcttaccccctggatggagctgctagaggtgaggacacctccatgaaccttgtgcaaaagtacctggagaactactatgacctgaagaaggatgtgaagcagtttgtgagaaggaaggattctggacctgtggtcaaaaagatcagggagatgcagaaattccttggactggaagtgacaggaaagctggactctgacactcttgaagtcatgagaaagcctagatgtggagtccctgatgtgggccatttcagaaccttccctggtattcctaagtggagaaagactcacctgacatacaggattgtgaactacactccagacctccctaaggatgcagtggacagtgcagtggaaaaggccttgaaagtctgggaggaagtgacccctctgacctttagtagactgtatgaaggtgaagctgatatcatgatctcctttgctgtgagagaacatggggacttttatccttttgatggccctgggaatgtcctggctcatgcttatgcccctggacctgggatcaatggagatgcccactttgatgatgatgagcagtggaccaaggacaccacaggtactaacctgttcctggtggcagcccatgagattggtcattccctgggactcttccattctgctaacactgaagccctgatgtaccctctgtaccactccctgacagacctcactagattcaggctgagccaggatgatatcaatggcattcagagcctctatggacccccacctgattccccagaaaccccactggtgcctacagagccagtgccaccagagcctggaactccagctaactgtgaccctgctctgagctttgatgctgtgtcaaccctgaggggagaaattctgattttcaaggacagacacttctggaggaagagcctcagaaagctggagcctgagctgcacctcatttccagtttctggccaagccttccatctggagtggatgctgcatatgaagtcacatccaaagacctggtgttcatctttaagggcaatcagttctgggctattagaggcaatgaagtcagggctggctaccctagaggcatccacaccctggggtttccccccactgtgagaaagattgatgcagccatcagtgacaaggaaaagaacaagacctacttctttgtggaggacaagtactggagatttgatgagaagaggaatagcatggaacctggcttccctaagcaaattgctgaggacttcccaggtattgactccaagattgatgcagtgtttgaggagtttggtttcttctacttcttcacagggtcatctcagctggaatttgaccccaatgctaagaaagtcactcacaccctcaagtccaactcctggctcaactgttga

(19) United States
(12) Patent Application Publication
(10) Pub. No.: US 2022/0362402 A1
(43) Pub. Date: Nov. 17, 2022

(54) COMPOSITIONS AND METHODS FOR OCULAR THERAPY

(71) Applicants: Exhaura, Ltd., Palo Alto (US); The Provost, Fellows, Foundation Scholars, and the other members of Board, of the College of the, Dublin (IE)

(72) Inventors: Jeffrey O'CALLAGHAN, Dublin (IE); Thomas W. CHALBERG, Jr., Palo Alto, CA (US); Matthew LAWRENCE, Palo Alto, CA (US); Annahita KERAVALA, Palo Alto, CA (US); Matthew CAMPBELL, Dublin (IE); Peter HUMPHRIES, Dublin (IE)

(21) Appl. No.: 17/765,368

(22) PCT Filed: Oct. 7, 2020
(86) PCT No.: PCT/US2020/054620

§ 371 Date: Mar. 30, 2022

Related U.S. Application Data
(60) Provisional application No. 62/912,427, filed on Oct. 8, 2019.

(51) Int. Cl. A61K 48/00; C12N 15/86; C12N 9/64

(52) U.S. Cl. CPC A61K 48/00; C12N 15/86; C12N 9/6491; C12Y 304/24017; C12N 2750/14143; C12N 2800/22; C12N 2830/42; C12N 2830/50

(57) ABSTRACT

Provided is a unit dose of recombinant adeno-associated virus (AAV) particles for expression of matrix metalloproteinase 3 (MMP-3). Further provided is a unit dose of recombinant MMP-3. Also provided are methods of use thereof, e.g., in transducing the corneal endothelium of a subject; reducing intraocular pressure in an eye of a subject; treating and/or preventing elevated intraocular pressure in a subject; and treating and/or preventing glaucoma in a subject. Subjects include primates.

### CROSS-REFERENCE TO RELATED APPLICATIONS

This application claims priority to U.S. Provisional Patent Application No. 62/912,427, filed Oct. 8, 2019, the disclosure of which is incorporated by reference herein in its entirety for all purposes.

### TECHNICAL FIELD

The present disclosure relates to ocular therapy, including the use of adeno-associated virus (AAV) vectors for delivery of a therapeutic gene.

### INCORPORATION OF THE SEQUENCE LISTING

The contents of the text file submitted electronically herewith are incorporated herein by reference in their entirety: A computer readable format copy of the Sequence Listing (filename: EXHA_004_01_WO_SeqList_ST25.txt, date recorded Oct. 7, 2020, file size 50 kb).

### BACKGROUND

Intraocular pressure (IOP) is maintained as a result of the balance between production of aqueous humor (AH) by the ciliary processes and hydrodynamic resistance to its outflow through the conventional outflow pathway comprising the trabecular meshwork (TM) and Schlemm's canal (SC). Elevated IOP, which can be caused by increased resistance to AH outflow, is a major risk factor for open-angle glaucoma. Matrix metalloproteinases (MMPs) contribute to conventional aqueous outflow homeostasis in their capacity to remodel extracellular matrices, which has a direct impact on aqueous outflow resistance and IOP. Decreased MMP-3 activity has been observed in human glaucomatous AH compared to age-matched normotensive control AH. Treatment with glaucomatous AH resulted in significantly increased transendothelial resistance of SC endothelial and TM cell monolayers and reduced monolayer permeability when compared to control AH, or supplemented treatment with exogenous MMP-3.

There remains an unmet need for improved compositions and methods for gene therapy or recombinant protein-based therapy for elevated IOP. The disclosure provides such novel compositions and methods to address and solve this need.

### SUMMARY

In an aspect, provided are compositions and methods for ocular therapy. In one aspect, compositions can be used to treat certain ocular diseases. In some aspects, compositions include nucleic acid and protein sequences for MMP-3.

In some aspects the disclosure provides, a unit dose comprising a plurality of recombinant adeno-associated virus of serotype 9 (rAAV9) particles, wherein each rAAV9 of the plurality of rAAV9 particles is non-replicating, and wherein each rAAV9 of the plurality of rAAV9 particles comprises a polynucleotide comprising, from 5′ to 3′: (a) a sequence encoding a 5′ inverted terminal repeat (ITR); (b) a sequence encoding a promoter; (c) a sequence encoding a human matrix metalloproteinase 3 (hMMP-3); (d) a sequence encoding a polyadenylation (polyA) signal; and (e) a sequence encoding a 3′ ITR; and wherein the unit dose comprises between 1×10¹⁰ vector genomes (vg) and 5×10¹² vg, inclusive of the endpoints, of rAAV9 particles.

In some embodiments, the unit dose is (i) sterile and (ii) comprises a pharmaceutically acceptable carrier. In some embodiments, each rAAV9 of the plurality of rAAV9 particles is a single-stranded AAV (ssAAV) vector. In some embodiments, each rAAV9 of the plurality of rAAV9 particles is a self-complementary AAV (scAAV) vector. In some embodiments, the promoter comprises a CMV promoter, and wherein the sequence encoding the CMV promoter comprises or consists of the sequence of SEQ ID NO: 6, SEQ ID NO: 7, or SEQ ID NO: 19, or a functional variant thereof, optionally having 80%, 90%, 95%, or 99% sequence identity thereto. In some embodiments, the sequence encoding human MMP-3 comprises or consists of a nucleotide sequence encoding the MMP-3 amino acid sequence of SEQ ID NO: 1, SEQ ID NO: 2, SEQ ID NO: 10 or SEQ ID NO: 22, or a functional variant thereof, optionally having 80%, 90%, 95%, or 99% sequence identity thereto. In some embodiments, the nucleotide sequence encoding the MMP-3 amino acid sequence comprises a wild-type nucleotide sequence. In some embodiments, the sequence encoding MMP-3 comprises or consists of the nucleotide sequence of SEQ ID NO: 9, SEQ ID NO: 23, SEQ ID NO: 24, SEQ ID NO: 25, SEQ ID NO: 26, or SEQ ID NO: 27, or shares at least 80%, 90%, 95%, 97%, 99% sequence identity to thereto. In some embodiments, the sequence encoding the 5′ ITR is derived from a 5′ ITR sequence of an AAV of serotype 2 (AAV2). In some embodiments, the sequence encoding the 5′ ITR comprises a sequence that is identical to a sequence of a 5′ ITR of an AAV2. In some embodiments, the sequence encoding the 5′ ITR comprises or consists of the nucleotide sequence of SEQ ID NO: 5, SEQ ID NO: 14, or SEQ ID NO: 15. In some embodiments, the sequence encoding the 3′ ITR is derived from a 3′ ITR sequence of an AAV2. In some embodiments, the sequence encoding the 3′ ITR comprises a sequence that is identical to a sequence of a 3′ ITR of an AAV2. In some embodiments, the sequence encoding the 3′ ITR comprises or consists of the nucleotide sequence of SEQ ID NO: 12 or any one of SEQ ID NOs: 16-18. In some embodiments, the sequence encoding the polyA signal comprises a human growth hormone (hGH) polyA sequence. In some embodiments, the sequence encoding the hGH polyA signal comprises the nucleotide sequence of SEQ ID NO: 11. In some embodiments, the polynucleotide further comprises a Kozak sequence. In some embodiments, the Kozak sequence comprises or consists of the nucleotide sequence of CGCCACCATG (SEQ ID NO: 21). In some embodiments, the polynucleotide comprises or consists of the sequence of (SEQ ID NO: VECTOR). In some embodiments, the rAAV9 particles comprise a viral Cap protein isolated or derived from an AAV serotype 9 (AAV9) Cap protein. In some aspects, the disclosure provides a unit dose comprising recombinant matrix metalloproteinase 3 (MMP-3) protein, wherein the unit dose comprises between 1 milligrams per milliliter (mg/mL) and 500 mg/mL, inclusive of the endpoints, of the recombinant MMP-3 protein; or between 0.1 nanograms (ng) and 10 ng, inclusive of the endpoints, of the recombinant MMP-3 protein. In some embodiments, the unit dose comprises about 10 ng/mL of the recombinant MMP-3 protein. In some embodiments, the recombinant MMP-3 protein is a human MMP-3 protein. In some embodiments, the recombinant MMP-3 protein has a polypeptide sequence that comprises or consist of the sequence of SEQ ID NO: 1, SEQ ID NO: 2, SEQ ID NO: 10 or SEQ ID NO: 22, or a functional variant or functional fragment thereof, optionally having 80%, 90%, 95%, or 99% sequence identity thereto.

In some aspects, the disclosure provides a method of transducing the corneal endothelium of a subject, comprising administering an effective amount of the unit dose described herein, wherein the subject is a primate. In some embodiments, administering the effective amount of the unit dose results in expression of MMP-3 in the aqueous humor of an eye of the subject at a measured concentration of between 0.01 ng/mL and about 10 ng/mL, inclusive of the endpoints, between 0.01 ng/mL and about 500 ng/mL, inclusive of the endpoints, or between 0.01 ng/mL and about 1000 ng/mL, inclusive of the endpoints. In some embodiments, the measured concentration is greater than or equal to 1 ng/mL. In some embodiments, the measured concentration is less than or equal to 10 ng/mL. In some embodiments, the measured concentration is 1-10 ng/mL, inclusive of the endpoints. In some embodiments, the measured concentration is at least 1-3 ng/mL, inclusive of the endpoints. In some embodiments, the expression of MMP-3 is maintained at least 21 days, 42 days, 56 days, or 66 days. In some embodiments, the expression of MMP-3 is maintained at least 90 days. In some embodiments, the expression of MMP-3 in aqueous humor is measured by Western Blot or ELISA. In some embodiments, the method increases outflow facility by at least 25% or by at least 30%. In some embodiments, the increase in outflow facility occurs within about 66 days of the administering step. In some embodiments, wherein the corneal thickness remains unchanged relative to corneal thickness in the subject before the administering step and/or relative to corneal thickness in a subject administered a control unit dose. In some embodiments, the method causes no inflammatory response. In some embodiments, the method results in serum levels of MMP-3 that are not elevated over a baseline level of MMP-3 in the serum of the subject. In some embodiments, the administering step comprises intracameral injection of the unit dose into at least one eye of the subject.

In some aspects, the disclosure provides a method of reducing intraocular pressure (IOP) in at least one eye of a subject, comprising administering an effective amount of the unit dose described herein, wherein the subject is a primate. In some embodiments, administering the effective amount of the unit dose results in expression of MMP-3 in the aqueous humor of an eye of the subject at a measured concentration of between 0.01 ng/mL and about 10 ng/mL, inclusive of the endpoints. In some embodiments, the measured concentration is greater than or equal to 1 ng/mL. In some embodiments, the measured concentration is less than or equal to 10 ng/mL. In some embodiments, the measured concentration is 1-10 ng/mL, inclusive of the endpoints. In some embodiments, the measured concentration is at least 1-3 ng/mL, inclusive of the endpoints. In some embodiments, the expression of MMP-3 is maintained at least 21 days, 42 days, 56 days, or 66 days. In some embodiments, the expression of MMP-3 is maintained at least 90 days. In some embodiments, the expression of MMP-3 is measured by Western Blot or ELISA. In some embodiments, the method increases outflow facility by at least 25% or by at least 30%. In some embodiments, the method reduces intraocular pressure (IOP). In some embodiments, the corneal thickness remains unchanged relative to corneal thickness in the subject before the administering step and/or relative to corneal thickness in a subject administered a control unit dose. In some embodiments, the method causes no inflammatory response. In some embodiments, the method results in serum levels of MMP-3 that are not elevated over a baseline level of MMP-3 in the serum of the subject. In some embodiments, the administering step comprises injection of the unit dose into the cornea of at least one eye of the subject. In some embodiments, the administering step comprises injection of the unit dose into the temporal cornea of at least one eye of the subject. In some embodiments, the administering step comprises intracameral injection of the unit dose into at least one eye of the subject.

In some aspects, the disclosure provides a method of treating and/or preventing elevated IOP and/or glaucoma in a subject in need thereof, comprising administering an effective amount of the unit dose described herein to the subject, wherein the subject is a primate.

In some aspects, the disclosure provides a method of transducing the corneal endothelium of a subject, comprising administering an effective amount of a unit dose comprising a plurality of recombinant adeno-associated virus of serotype 9 (rAAV9) particles to the subject, wherein the subject is a primate; wherein each rAAV9 of the plurality of rAAV9 particles is non-replicating; wherein each rAAV9 of the plurality of rAAV9 particles is a single-stranded AAV (ssAAV); wherein each rAAV9 of the plurality of rAAV9 particles comprises a polynucleotide comprising, from 5′ to 3′: (a) a sequence encoding a 5′ inverted terminal repeat (ITR); (b) a sequence encoding a promoter; (c) a sequence encoding a matrix metalloproteinase 3 (MMP-3); (d) a sequence encoding a polyadenylation (polyA) signal; and (e) a sequence encoding a 3′ ITR; and wherein the unit dose comprises (i) between 1×10¹⁰ vector genomes (vg) and 5×10¹² vg, inclusive of the endpoints, of rAAV9 particles; or (ii) about 1×10¹¹ vector genomes (vg) per milliliter (mL) to 1×10¹⁴ vg/mL of rAAV9 particles; and wherein administering the effective amount of the unit dose results in expression of MMP-3 in the aqueous humor of an eye of the subject at a measured concentration of between 0.01 ng/mL and about 10 ng/mL, inclusive of the endpoints. In some embodiments, the sequence encoding MMP-3 comprises or consists of the nucleotide sequence of SEQ ID NO: 9, SEQ ID NO: 23, SEQ ID NO: 24, SEQ ID NO: 25, SEQ ID NO: 26, or SEQ ID NO: 27, or shares at least 80%, 90%, 95%, 97%, 99% sequence identity to thereto.

In some aspects, the disclosure provides a method of transducing the corneal endothelium of a subject, comprising administering an effective amount of a unit dose comprising a plurality of recombinant adeno-associated virus of serotype 9 (rAAV9) particles to the subject, wherein the subject is a primate; wherein each rAAV9 of the plurality of rAAV9 particles is non-replicating; wherein each rAAV9 of the plurality of rAAV9 particles is a single-stranded AAV (ssAAV); wherein each rAAV9 of the plurality of rAAV9 particles comprises a polynucleotide comprising, from 5′ to 3′: (a) a sequence encoding a 5′ inverted terminal repeat (ITR); (b) a sequence encoding a promoter; (c) a sequence encoding a transgene; (d) a sequence encoding a polyadenylation (polyA) signal; a (e) a sequence encoding a 3′ ITR.

In some aspects, the disclosure provides a gene therapy vector comprising an expression cassette comprising a transgene encoding a human matrix metalloproteinase 3 (hMMP-3) or a functional variant thereof, optionally operatively linked to a promoter, wherein the transgene is optimized for expression in a human host cell. In some embodiments, the human host cell is a human corneal endothelial cell. In some embodiments, the transgene shares at least 80% identity, at least 85% identity, at least 90% identity, at least 95% identity, at least 97% identity, or at least 99% identity to a sequence selected from SEQ ID NOs: 23-27. In some embodiments, the transgene comprises a sequence selected from SEQ ID NOs: 23-27. In some embodiments, the transgene shares at least 95% identity to SEQ ID NO: 23 or is identical to SEQ ID NO: 23. In some embodiments, the transgene shares at least 95% identity to SEQ ID NO: 24 or is identical to SEQ ID NO: 24. In some embodiments, the transgene shares at least 95% identity to SEQ ID NO: 25 or is identical to SEQ ID NO: 25. In some embodiments, the transgene shares at least 95% identity to SEQ ID NO: 26 or is identical to SEQ ID NO: 26. In some embodiments, the transgene shares at least 95% identity to SEQ ID NO: 27 or is identical to SEQ ID NO: 27. In some embodiments, the vector is an adeno-associated virus (AAV) vector. In some embodiments, the AAV vector is an AAV9 vector. In some embodiments, the AAV vector is a single-stranded AAV (ssAAV) vector. In some embodiments, the AAV vector is a self-complementary AAV (ssAAV) vector.

In some aspects, the disclosure provides a pharmaceutical composition comprising the gene therapy vector described herein.

In some aspects, the disclosure provides a method of treating and/or preventing elevated IOP and/or glaucoma in a subject in need thereof, comprising administering an effective amount of the gene therapy vector described or the pharmaceutical composition described to the subject, wherein the subject is a primate.

In some aspects, the disclosure provides a polynucleotide, comprising a transgene encoding a human matrix metalloproteinase 3 (hMMP-3) or a functional variant thereof, wherein the transgene is optimized for expression in a human host cell.

In some embodiments, the polynucleotide comprises a promoter operatively linked to the transgene. In some embodiments, the human host cell is a human corneal endothelial cell. In some embodiments, the transgene shares at least 80% identity, at least 85% identity, at least 90% identity, at least 95% identity, at least 97% identity, or at least 99% identity to a sequence selected from SEQ ID NOs: 23-27. In some embodiments, the transgene comprises a sequence selected from SEQ ID NOs: 23-27. In some embodiments, the transgene shares at least 95% identity to SEQ ID NO: 23 or is identical to SEQ ID NO: 23. In some embodiments, the polynucleotide comprises adeno-associated virus (AAV) terminal repeats (ITRs) flanking the transgene. In some embodiments, the polynucleotide is an isolated polynucleotide.

In some embodiments, the transgene shares at least 95% identity to SEQ ID NO: 24 or is identical to SEQ ID NO: 24. In some embodiments, the transgene shares at least 95% identity to SEQ ID NO: 25 or is identical to SEQ ID NO: 25. In some embodiments, the transgene shares at least 95% identity to SEQ ID NO: 26 or is identical to SEQ ID NO: 26. In some embodiments, the transgene shares at least 95% identity to SEQ ID NO: 27 or is identical to SEQ ID NO: 27. In some embodiments, the polynucleotide comprises adeno-associated virus (AAV) terminal repeats (ITRs) flanking the transgene. In some embodiments, the polynucleotide is an isolated polynucleotide.

In some aspects, the disclosure provides an isolated cell, comprising a polynucleotide described herein.

In some aspects, the disclosure provides a pharmaceutical composition, comprising the polynucleotide described herein.

Further aspects and embodiments of the invention are provided by the Detailed Description that follows.

### BRIEF DESCRIPTION OF THE DRAWINGS

FIG. 1 shows a box and whisker plot of recombinant human matrix metalloproteinase 3 (rhMMP3) in the aqueous humor of monkeys after intraocular infusion of rhMMP3.

FIG. 2A shows a plot of relative difference in outflow facility between treated and contralateral eyes in 17 primate subjects (labeled 1-17).

FIG. 2B shows a plot of relative difference in outflow facility between treated and contralateral eyes against measured rhMMP3 concentration in the aqueous humor (AH) of the primate subject.

FIG. 3A shows a fluorescence micrograph of the cornea of a primate subject treated with an intracameral injection of self-complementary AAV serotype 9 (scAAV9-EGFP).

FIG. 3B shows a fluorescence micrograph of the cornea of a primate subject treated with an intracameral injection of single stranded AAV (ssAAV9-EGFP).

FIG. 3C shows a Z-stack reconstruction of fluorescence micrographs of the cornea of a primate subject treated with an intracameral single stranded AAV (ssAAV9-EGFP).

FIG. 4 shows a plot of average concentration of MMP-3 in the aqueous humor of eyes of primate subjects intracamerally injected with AAV9-CMV-MMP3 or AAV9-CMV-eGFP.

FIG. 5A shows a plot of intraocular pressure (IOP) in millimeters mercury (mmHg) over time (days) in the eyes of primate subjects intracamerally injected with AAV9-CMV-MMP3 or saline control.

FIG. 5B shows a plot of change in intraocular pressure (ATOP) in millimeters mercury (mmHg), in the eyes of primate subjects intracamerally injected with AAV9-CMV-MMP3, against the expression level of MMP-3 in the aqueous humor observed in that eye (in ng/mL).

FIG. 6A shows a plot of mean corneal thickness (μm) measured by pachymetry over time (days) in the eyes of primate subjects intracamerally injected with AAV9-CMV-MMP3 or saline control.

FIG. 6B shows a plot of mean corneal thickness (μm) measured by specular microscopy over time (days) in the eyes of primate subjects intracamerally injected with AAV9-CMV-MMP3 or saline control.

FIG. 7 shows a graph of serum levels of MMP-3 determined by ELISA in treated (bottom line) and vehicle control (top line) subjects.

FIG. 8A shows a chart of intraocular pressure (IOP) over time (weeks) in dexamethasone-treated [DEX(+)] animals intracamerally injected with adeno-associated vector inducibly expressing MMP-3 or GFP control.

FIG. 8B shows a chart of intraocular pressure (IOP) over time (weeks) in control [DEX(−)] animals intracamerally injected with adeno-associated vector inducibly expressing MMP-3 or GFP control.

FIGS. 9A-9B show dot-box plots of the change in IOP from baseline (Pre-injection) to the final measurement (DEX Week 4) for AAV-iMMP-3 treated eyes (left) and contralateral AAV-iGFP controls (right) in both DEX treated (DEX (+), FIG. 9A) and the cyclodextrin control group (DEX (−), FIG. 9B). MMP-3 significantly reduces IOP in the hypertensive model only.

FIGS. 9C-9D show dot-box plots of IOP at Week 4 for AAV-iMMP-3 treated eyes (left) and contralateral AAV-iGFP controls (right) in both DEX treated (DEX (+), FIG. 9C) and the cyclodextrin control group (DEX (−), FIG. 9D).

FIG. 10A shows a cello plot depicting paired analysis between AAV-iMMP-3 and AAV-iGFP treated eyes in the DEX treated cohort of outflow facility. Average percentage facility difference is denoted by the white line, with the dark blue shading as the 95% CI of the mean. Individual data points are plotted along with their own 95% CIs.

FIG. 10B shows a cello plot depicting paired analysis between AAV-iMMP-3 and AAV-iGFP treated eyes in the cyclodextrin control group of outflow facility. Average percentage facility difference is denoted by the white line, with the dark blue shading as the 95% CI of the mean. Individual data points are plotted along with their own 95% CIs.

FIG. 11 shows a plot of percent (%) optically empty space in treated (AAV-iMMP-3) and vector control (AAV-iGFP) eyes

FIG. 12A shows IOP of AAV-iMMP-3 (blue) and AAV-iGFP (red) treated eyes in mice transgenic for human myocilin Y437H.

FIG. 12B shows IOP of AAV-iMMP-3 (blue) and AAV-iGFP (red) treated eyes in wild-type mice.

FIGS. 13A-13B show dot-box plots of the change in IOP from baseline (Pre-injection) to the final measurement for AAV-iMMP-3 treated eyes (left) and contralateral AAV-iGFP controls (right) in both transgenic model (MYOC(+), FIG. 13A) and the control group (MYOC(−), FIG. 13B). MMP-3 significantly reduces IOP in the MYOC(+) model only.

FIGS. 13C-13D show dot-box plots of final IOP for AAV-iMMP-3 treated eyes (left) and contralateral AAV-iGFP controls (right) in both transgenic model (MYOC(+), FIG. 13C) and the control group (MYOC(−), FIG. 13D).

FIG. 14A shows a cello plot depicting paired analysis between AAV-iMMP-3 and AAV-iGFP treated eyes in MYOC(+) animals of outflow facility.

FIG. 14B shows a cello plot depicting paired analysis between AAV-iMMP-3 and AAV-iGFP treated eyes in MYOC(−) animals of outflow facility.

FIG. 15 shows a bar chart depicting the amount of recombinant MMP-3 produced by HEK293 cells transfected with native and codon optimized MMP-3 sequences.

FIG. 16 shows a bar chart depicting the amount of recombinant MMP-3 produced by HCEC cells transfected with native and codon optimized MMP-3 sequences.

FIG. 17 shows a bar chart depicting the amount of recombinant MMP-3 produced in HCEC cells that were transduced by an AAV9 viral vector encoding native or codon optimized MMP-3 sequences.

FIG. 18 shows a bar chart depicting the normalized amount of recombinant MMP-3 produced in HCEC cells that were transduced by an AAV9 viral vector encoding native or codon optimized MMP-3 sequences.

FIG. 19 shows an immunoblot showing the amount of recombinant proMMP-3 and active MMP-3 produced in HCEC cells that were transduced by an AAV9 viral vector encoding native or codon optimized MMP-3 sequences.

FIG. 20 shows a bar chart depicting the amount of MMP-3 protease activity in the media of HCEC cells that were transduced by an AAV9 viral vector encoding native or codon optimized MMP-3 sequences.

FIG. 21A shows a cello plot depicting outflow facility (nl/min/mmHg) values of vehicle and MMP-3 treated human eyes one hour after an infusion of 5 ng/ml MMP-3 into the anterior chamber. FIG. 21B shows a cello plot depicting the percent difference between vehicle and experimental pairs of human eyes.

FIG. 22A-22C shows a sequence alignment of optimized polynucleotide sequences encoding MMP-3, according to an embodiment.

SEQUENCE LISTING

| SEQ ID NO: | Description |
|---|---|
| 1 | Full length human MMP-3 amino acid sequence |
| 2 | Recombinant human MMP-3 amino acid |
| | sequence (lacking pro-peptide domain) |
| 3 | Full length vector (not including backbone) |
| 4 | Expression cassette (not including ITRs) |
| 5 | AAV2 ITR 1-130 130bp |
| 6 | CMV enhancer 210-513 304bp |
| 7 | CMV promoter 514-716 203bp |
| 8 | Human beta-globin Intron 809-1301 493bp |
| 9 | Human MMP3 1332-2765 1434bp (nucleotide) |
| 10 | Human MMP3 1332-2765 1434bp (amino acid) |
| 11 | hGH poly(A) signal 2847-3323 477bp |
| 12 | AAV2 ITR (inverted) 3363-3503 141bp |
| 13 | AAV9 capsid sequence |
| 14 | 5′ ITR |
| 15 | 5′ ITR |
| 16 | 3′ ITR |
| 17 | 3′ ITR |
| 18 | 3′ ITR |
| 19 | CMV enhancer/promoter |
| 20 | hGH polyA |
| 21 | Kozak |
| 22 | Full length human MMP-3 amino acid |
| | sequence (without signal sequence) |
| 23 | MMP3 Opt 1 |
| 24 | MMP3 Opt 2 |
| 25 | MMP3 Opt 3 |
| 26 | Native MMP3 CpG depleted |
| 27 | MMP3 Opt 3 CpG depleted |

### DETAILED DESCRIPTION

The present disclosure relates generally to therapeutic use of recombinant proteins and gene therapy vectors, particularly adeno-associated virus (AAV) vectors, in treatment of ocular conditions in primate subjects (e.g., monkeys, apes, and humans); and to the therapeutic delivery of genes including proteinases and without limitation matrix metalloproteinases, such as matrix metalloproteinase 3 (MMP-3), to the eye by use of AAV vectors, or by directing injection of recombinant protein, e.g. recombinant human MMP-3 (rhMMP-3). Disclosed herein are AAV vectors that effectively transduce structures in the anterior chamber of the eye, including the corneal endothelium of a subject, increasing outflow facility in the eye of a subject, and/or reducing the intraocular pressure in the eye of a subject. Further disclosed herein are unit doses of AAV vectors at concentrations determined to be effective in decreasing and/or preventing elevated intraocular pressure in a subject. Further disclosed herein are unit doses of rhMMP-3 at concentrations determined to be effective in decreasing and/or preventing elevated intraocular pressure in a subject. In some embodiments, the subject in a primate.

Intracameral inoculation of AAV-2/9 containing a CMV-driven murine MMP-3 gene (AAV-muMMP-3) into wild type mice resulted in efficient transduction of corneal endothelium and an increase in aqueous concentration and activity of muMMP-3. O'Callaghan et al. Hum. Mol. Genet. 26:1230-1246 (2017). However, determination of effective dosing strategies for primates is hindered by differences in the MMP-3 sequence between mice and primates, the size difference between mice and primates, and differences in the physiology and cell biology of the corneal endothelium. Prior efforts to transduce the corneal epithelium of primates have shown that transgene expression disappears within 70 days and inflammation occurs. Buie et al. Investigative Ophthalmology & Visual Science 51:236-48 (2010). Prior efforts have not characterized intracameral delivery (i.e., delivery to the anterior chamber of the eye) of human MMP-3 (referred to herein as hMMP-3 or alternatively huMMP-3), either as recombinant protein (rhMMP-3) or gene therapy vector encoding the hMMP-3 gene.

### Unit Dose

Provided herein are unit doses of a plurality of recombinant adeno-associated virus (AAV) particles and unit doses of recombinant human matrix metalloproteinase 3 (rhMMP-3) protein. As used herein, a “unit dose” refers to an amount of a therapeutic composition administered to a subject in a single dose. A single dose may be administered in one injection or multiple injections within a predetermined period of time, e.g. 1 hours, 2 hours, 12 hours, or 24 hours.

A unit dose may be defined by the amount, concentration, and/or volume of a therapeutic composition (e.g. AAV particles or recombinant proteins). For AAV, the amount may be expressed in terms of genome particles (gp), DNase resistant particles (DRP), or vector genomes (vg). As used herein, “vector genomes” refers to a number of particles determined by quantitative polymerase chain reaction (qPCR) titration against a reference standard. Unencapsidated DNA is removed using DNAse, and viral proteins are then degraded by incubation proteinase K. Samples are diluted and run in quadruplicate using a master mix containing 2X TAQMAN Universal Master Mix, 20X TAQMAN Gene Expression Assay probes targeting the polynucleotide of the viral particle (e.g. the polynucleotide encoding MMP-3), and RNase-free water. Samples are compared against a standard curve of known concentration and reference standards. The qPCR reaction is performed on a STEPONEPLUS (Applied Biosystems®) instrument for 40 cycles of denaturing and annealing, with a prior 10-minute polymerase activation step. Data is analyzed on the instrument. Plasmid DNA containing part or all of the viral genome may be used as the reference standard. For example, pcDNA3-EGFP may be used as the reference standard for AAV particles comprising a polynucleotide comprising a sequence encoding EGFP. For AAV particles comprising a polynucleotide comprising a sequence encoding MMP-3, the plasmid used to generate the AAV particles may be used as the reference standard for determining the titer of the AAV particles by qPCR. Primers for qPCR are selected to amplify both the reference standard and the viral genome.

The concentrations of the AAV particles may be expressed as a titer, that is an amount divided by a volume, e.g. vector genomes per milliliter (vg/mL), gp/mL, and DRP/mL. In some embodiments, unit dose comprises a concentration of rAAV9 particles between 1×10⁹ vector genomes per milliliter (vg/mL) and 5×10¹³ vg/mL, inclusive of the endpoints. In some embodiments, the unit does comprises a concentration of rAAV9 particles between 1×10⁹ vg/mL to 2.5×10⁹ vg/mL, 2.5×10⁹ vg/mL to 5×10⁹ vg/mL, 5×10⁹ vg/mL to 7.5×10⁹ vg/mL, 7.5×10⁹ vg/mL to 1×10¹⁰ vg/mL, 1×10¹⁰ vg/mL to 2.5×10¹⁰ vg/mL, 2.5×10¹⁰ vg/mL to 5×10¹⁰ vg/mL, 5×10¹⁰ vg/mL to 7.5×10¹⁰ vg/mL, 7.5×10¹⁰ vg/mL to 1×10¹¹ vg/mL, 1×10¹¹ vg/mL to 2.5×10¹¹ vg/mL, 2.5×10¹¹ vg/mL to 5×10¹¹ vg/mL, 5×10¹¹ vg/mL to 7.5×10¹¹ vg/mL, 7.5×10¹¹ vg/mL to 1×10¹² vg/mL, 1×10¹² vg/mL to 2.5×10¹² vg/mL, 2.5×10¹² vg/mL to 5×10¹² vg/mL, 5×10¹² vg/mL to 7.5×10¹² vg/mL, 7.5×10¹² vg/mL to 1×10¹³ vg/mL, 1×10¹³ vg/mL to 2.5×10¹³ vg/mL, or 2.5×10¹³ vg/mL to 5×10¹³ vg/mL.

In some embodiments, the unit dose comprises a concentration of rAAV9 particles of about 1×10⁹ vg/mL, about 2.5×10⁹ vg/mL, about 5×10⁹ vg/mL, about 7.5×10⁹ vg/mL, about 1×10¹⁰ vg/m, about 2.5×10¹⁰ vg/mL, about 5×10¹⁰ vg/mL, about 7.5×10¹⁰ vg/mL, about 1×10¹¹ vg/mL, about 2.5×10¹¹ vg/mL, about 5×10¹¹ vg/mL, about 7.5×10¹¹ vg/mL, about 1×10¹² vg/mL, about 2.5×10¹² vg/mL, about 5×10¹² vg/mL, about 7.5×10¹² vg/mL, about 1×10¹³ vg/mL, about 2.5×10¹³ vg/mL, or about 5×10¹³ vg/mL.

In some embodiments, the unit dose comprises between 1×10⁷ vector genomes (vg) and 5×10¹² vg, inclusive of the endpoints, of rAAV9 particles. In some embodiments, the unit does comprises between 1×10⁷ vg and 2.5×10⁷ vg, between 2.5×10⁷ vg and 5×10⁷ vg, between 5×10⁷ vg and 7.5×10⁷ vg, between 7.5×10⁷ vg and 1×10⁸ vg, between 1×10⁸ vg and 2.5×10⁸ vg, between 2.5×10⁸ vg and 5×10⁸ vg, between 5×10⁸ vg and 7.5×10⁸ vg, between 7.5×10⁸ vg and 1×10⁹ vg, between 1×10⁹ vg and 2.5×10⁹ vg, between 2.5×10⁹ vg and 5×10⁹ vg, between 5×10⁹ vg and 7.5×10⁹ vg, between 7.5×10⁹ vg and 1×10¹⁰ vg, between 1×10¹⁰ vg and 2.5×10¹⁰ vg, between 2.5×10¹⁰ vg and 5×10¹⁰ vg, between 5×10¹⁰ vg and 7.5×10¹⁰ vg, between 7.5×10¹⁰ vg and 1×10¹¹ vg, between 1×10¹¹ vg and 2.5×10¹¹ vg, between 2.5×10¹¹ vg and 5×10¹¹ vg, between 5×10¹¹ vg and 7.5×10¹¹ vg, between 7.5×10¹¹ vg and 1×10¹² vg, between 1×10¹² vg and 2.5×10¹² vg, or between 2.5×10¹² vg and 5×10¹² vg of rAAV9 particles.

In some embodiments, the unit dose comprises about 1×10⁷ vg, about 2.5×10⁷ vg, about 5×10⁷ vg, about 7.5×10⁷ vg, about 1×10⁸ vg, about 2.5×10⁸ vg, about 5×10⁸ vg, about 7.5×10⁸ vg, about 1×10⁹ vg, about 2.5×10⁹ vg, about 5×10⁹ vg, about 7.5×10⁹ vg, about 1×10¹⁰ vg, about 2.5×10¹⁰ vg, about 5×10¹⁰ vg, about 7.5×10¹⁰ vg, about 1×10¹¹ vg, about 2.5×10¹¹ vg, about 5×10¹¹ vg, about 7.5×10¹¹ vg, about 1×10¹² vg, about 2.5×10¹² vg, or about 5×10¹² vg of rAAV9 particles.

In some embodiments, unit dose comprises a concentration of rAAV particles between 1×10⁹ vector genomes per milliliter (vg/mL) and 5×10¹³ vg/mL, inclusive of the endpoints. In some embodiments, the unit does comprises a concentration of rAAV particles between 1×10⁹ vg/mL to 2.5×10⁹ vg/mL, 2.5×10⁹ vg/mL to 5×10⁹ vg/mL, 5×10⁹ vg/mL to 7.5×10⁹ vg/mL, 7.5×10⁹ vg/mL to 1×10¹⁰ vg/mL, 1×10¹⁰ vg/mL to 2.5×10¹⁰ vg/mL, 2.5×10¹⁰ vg/mL to 5×10¹⁰ vg/mL, 5×10¹⁰ vg/mL to 7.5×10¹⁰ vg/mL, 7.5×10¹⁰ vg/mL to 1×10¹¹ vg/mL, 1×10¹¹ vg/mL to 2.5×10¹¹ vg/mL, 2.5×10¹¹ vg/mL to 5×10¹¹ vg/mL, 5×10¹¹ vg/mL to 7.5×10¹¹ vg/mL, 7.5×10¹¹ vg/mL to 1×10¹² vg/mL, 1×10¹² vg/mL to 2.5×10¹² vg/mL, 2.5×10¹² vg/mL to 5×10¹² vg/mL, 5×10¹² vg/mL to 7.5×10¹² vg/mL, 7.5×10¹² vg/mL to 1×10¹³ vg/mL, 1×10¹³ vg/mL to 2.5×10¹³ vg/mL, or 2.5×10¹³ vg/mL to 5×10¹³ vg/mL.

In some embodiments, the unit dose comprises a concentration of rAAV particles of about 1×10⁹ vg/mL, about 2.5×10⁹ vg/mL, about 5×10⁹ vg/mL, about 7.5×10⁹ vg/mL, about 1×10¹⁰ vg/m, about 2.5×10¹⁰ vg/mL, about 5×10¹⁰ vg/mL, about 7.5×10¹⁰ vg/mL, about 1×10¹¹ vg/mL, about 2.5×10¹¹ vg/mL, about 5×10¹¹ vg/mL, about 7.5×10¹¹ vg/mL, about 1×10¹² vg/mL, about 2.5×10¹² vg/mL, about 5×10¹² vg/mL, about 7.5×10¹² vg/mL, about 1×10¹³ vg/mL, about 2.5×10¹³ vg/mL, or about 5×10¹³ vg/mL.

In some embodiments, the unit dose comprises between 1×10⁷ vector genomes (vg) and 5×10¹² vg, inclusive of the endpoints, of rAAV particles. In some embodiments, the unit dose comprises between 1×10⁷ vg and 2.5×10⁷ vg, between 2.5×10⁷ vg and 5×10⁷ vg, between 5×10⁷ vg and 7.5×10⁷ vg, between 7.5×10⁷ vg and 1×10⁸ vg, between 1×10⁸ vg and 2.5×10⁸ vg, between 2.5×10⁸ vg and 5×10⁸ vg, between 5×10⁸ vg and 7.5×10⁸ vg, between 7.5×10⁸ vg and 1×10⁹ vg, between 1×10⁹ vg and 2.5×10⁹ vg, between 2.5×10⁹ vg and 5×10⁹ vg, between 5×10⁹ vg and 7.5×10⁹ vg, between 7.5×10⁹ vg and 1×10¹⁰ vg, between 1×10¹⁰ vg and 2.5×10¹⁰ vg, between 2.5×10¹⁰ vg and 5×10¹⁰ vg, between 5×10¹⁰ vg and 7.5×10¹⁰ vg, between 7.5×10¹⁰ vg and 1×10¹¹ vg, between 1×10¹¹ vg and 2.5×10¹¹ vg, between 2.5×10¹¹ vg and 5×10¹¹ vg, between 5×10¹¹ vg and 7.5×10¹¹ vg, between 7.5×10¹¹ vg and 1×10¹² vg, between 1×10¹² vg and 2.5×10¹² vg, or between 2.5×10¹² vg and 5×10¹² vg of rAAV particles.

In some embodiments, the unit dose comprises about 1×10⁷ vg, about 2.5×10⁷ vg, about 5×10⁷ vg, about 7.5×10⁷ vg, about 1×10⁸ vg, about 2.5×10⁸ vg, about 5×10⁸ vg, about 7.5×10⁸ vg, about 1×10⁹ vg, about 2.5×10⁹ vg, about 5×10⁹ vg, about 7.5×10⁹ vg, about 1×10¹⁰ vg, about 2.5×10¹⁰ vg, about 5×10¹⁰ vg, about 7.5×10¹⁰ vg, about 1×10¹¹ vg, about 2.5×10¹¹ vg, about 5×10¹¹ vg, about 7.5×10¹¹ vg, about 1×10¹² vg, about 2.5×10¹² vg, or about 5×10¹² vg of rAAV particles.

In some embodiments, the unit dose comprises a volume between 10 μl and 200 μl, or between 20 μl and 100 μl. In some embodiments, the unit dose is about 50 μl, about 60 μl, about 70 μl, about 80 μl, about 90 μl, or about 100 μl.

### Vector and AAV

The term “vector” is used here in its most general meaning and comprises any intermediary vehicle for a nucleic acid which enables said nucleic acid, for example, to be introduced into prokaryotic and/or eukaryotic cells and, where appropriate, to be integrated into a genome. Vectors may be replicated and/or expressed in the cells. Vectors comprise plasmids, phagemids, bacteriophages and viral genomes.

As applied to AAV, a “vector” refers both to a plasmid comprising a polynucleotide encoding the viral DNA genome and to the viral particle produced by packing the viral DNA genome into a recombinant AAV particle including capsid and other accessory proteins.

As used herein, the term “AAV” is an abbreviation for adeno-associated virus or a recombinant vector thereof. Adeno-associated virus is a single-stranded DNA parvovirus that grows only in cells in which certain functions are provided by a co-infecting helper virus. General information and reviews of AAV can be found in, for example, Carter, Handbook of Parvoviruses, 1:169-228 (1989), and Berns, Virology, 1743-1764 (1990).

As used herein, an “AAV vector” or “rAAV vector” refers to a recombinant vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing Rep and Cap gene products.

As used herein, an “AAV virion” or “AAV viral particle” or “AAV vector particle” refers to a viral particle composed of at least one AAV capsid protein and an encapsulated polynucleotide AAV vector. As used herein, if the particle comprises a heterologous polynucleotide (i.e. a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector with a vector genome contained within an AAV vector particle.

Adeno-associated virus (AAV) is a replication-deficient parvovirus, the single-stranded DNA genome of which is about 4.7 kb in length including two 145 nucleotide inverted terminal repeat (ITRs). There are multiple known variants of AAV, also sometimes called serotypes when classified by antigenic epitopes. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in U.S. Pat. No. 9,434,928, incorporated herein by reference. The sequence of ancestral AAVs including AAV.Anc80, AAV.Anc80L65 and their derivatives are described in WO2015054653A2 and Wang et al. Single stranded adeno-associated virus achieves efficient gene transfer to anterior segment in the mouse eye. PLoS One. 12(8):e0182473 (2017). Cis-acting sequences directing viral DNA replication, encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p9), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka et al., Current Topics in Microbiology and Immunology, 158:97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

In some cases, the AAV vectors and particles of the disclosure are used to deliver a polynucleotide sequence to the corneal endothelium of a primate. Polynucleotide sequences that can be delivered using the AAV vectors and particles of the disclosure include protein-coding and RNA-coding genes. In some embodiments, the polynucleotide of the AAV vector encodes one or more (or all) components of a gene editing system. The disclosure further provides multi-vector systems. In some embodiments, the vector systems is a split vector system in which a gene larger than about 4.5 kB is provided in two vectors that are joined by intracellular homologous recombination to form a single coding polynucleotide. The disclosure is not to be read as limiting the invention solely to delivery of matrix metalloproteinases. The invention is limited only by the claims.

### AAV9

As used herein a recombinant adeno-associated virus of serotype 9 (rAAV9) particle refers to genetically engineered AAV particle having a capsid protein that shares at least 85%, 90%, 95%, 96%, 97%, 98%, or 99% identity to the capsid protein of wild-type AAV9 and retains one or more functional properties of AAV9. Illustrative AAV9 capsid sequences are provided in U.S. Pat. Nos. 7,906,111 and 9,737,618. In some embodiments, the rAAV9 particle comprises a capsid protein that shares at least 90%, 95%, 96%, 97%, 98%, or 99% identity to amino acids 1 to 736, 138 to 736, or 203 to 736 of SEQ ID NO: 13.

In some cases, the rAAV vector is of the serotype AAV1, AAV2, AAV3b, AAV4, AAV5, AAV6, AAV7, AAV8, AAV9, AAV10, AAV11, AAV12, AAV13, or LK03, Anc80L65. Anc80L65 is described in Sharma et al. Transduction efficiency of AAV 2/6, 2/8 and 2/9 vectors for delivering genes in human corneal fibroblasts. PLoS ONE 12(8): e0182473 (2017). LKO3 is described in Lisowski et al., Selection and evaluation of clinically relevant AAV variants in a xenograft liver model, Nature. 2014 Feb. 20; 506(7488): 382-386.

Production of pseudotyped rAAV is disclosed in, for example, WO 2001/083692. Other types of rAAV variants, for example rAAV with capsid mutations, are also contemplated. See, for example, Marsic et al., Mol. Ther., 22(11):1900-1909 (2014). In some cases, the rAAV vector is of the serotype AAV9. In some embodiments, said rAAV vector is of serotype AAV9 and comprises a single stranded genome. Such AAV are termed “single stranded AAV” or “ssAAV.” In some embodiments, said rAAV vector is of serotype AAV9 and comprises a self-complementary genome. Such AAV are termed “self-complementary AAV” or “scAAV.” The present inventors have unexpectedly determined that, in some cases, ssAAV transduces the corneal endothelium of primates with higher efficiency that scAAV.

In some embodiments, each of the rAAV9 particles comprise a viral Cap protein isolated or derived from an AAV serotype 9 (AAV9) Cap protein.

### Polynucleotide

The AAV particles of the disclosure comprise at least one polynucleotide or exactly one polynucleotide. The polynucleotides of the disclosure comprise from 5′ to 3′, (a) a sequence encoding a 5′ inverted terminal repeat (ITR); (b) a sequence encoding a promoter; (c) a sequence encoding a transgene; (d) a sequence encoding a polyadenylation (polyA) signal; (e) a sequence encoding a 3′ ITR.

### Inverted Terminal Repeats

As used herein, “inverted terminal repeat sequences” or “ITRs” refer to analogous self-annealing segments at the termini of the AAV genome. In the context of a plasmid, the ITRs flank the DNA segment that is transcribed to form the AAV genome. The ITRs of the disclosure include any AAV ITR, including a wild type AAV ITR or a synthetic sequence that functions as an ITR for the AAV vector. In some embodiments, a rAAV vector comprises ITR sequences of AAV2. In some embodiments, the rAAV vector comprises an AAV2 genome, such that the rAAV vector is an AAV-2/9 vector, an AAV-2/6 vector, or an AAV-2/8 vector. Other combinations of genome and serotype are contemplated by the present disclosure, including, without limitation, those described in Sharm et al. Transduction efficiency of AAV 2/6, 2/8 and 2/9 vectors for delivering genes in human corneal fibroblasts. Brain Res Bull. 81:273-78 (2010).

In some embodiments, the sequence encoding the 5′ ITR is derived from a 5′ ITR sequence of an AAV of serotype 2 (AAV2). In some embodiments, the sequence encoding the 5′ ITR comprises a sequence that is identical to a sequence of a 5′ ITR of an AAV2. In some embodiments, the ITR is a heterologous or synthetic ITR that functions as an ITR. In some embodiments, the sequence encoding the 5′ ITR comprises or consists of the nucleotide sequence of SEQ ID NO: 5, SEQ ID NO: 14, or SEQ ID NO: 15.

In some embodiments, the sequence encoding the 3′ ITR is derived from a 3′ ITR sequence of an AAV2. In some embodiments, the sequence encoding the 3′ ITR comprises a sequence that is identical to a sequence of a 3′ ITR of an AAV2. In some embodiments, the ITR is a heterologous or synthetic ITR that functions as an ITR. In some embodiments, the sequence encoding the 3′ ITR comprises or consists of the nucleotide sequence of SEQ ID NO: 12 or any one of SEQ ID NOS: 16-18.

### Promoter

In some embodiments, the polynucleotide of the AAV particle may comprise a promoter, i.e., at least one promoter. In some embodiments, the polynucleotide comprises two promoters. In some embodiments, the polynucleotide comprises one promoter. In some embodiments, each promoter is independently selected from the group consisting of cytomegalovirus (CMV) promoter, a CAG promoter, an SV40 promoter, an SV40/CD43 promoter, and a MND promoter. A CAG promoter is a promoter sequence comprised of the CMV enhancer and portions of the chicken beta-actin promoter and the rabbit beta-globin gene. An SV40/CD43 promoter is a promoter sequence comprising portions of the SV40 promoter and CD43 promoter. An MND promoter is a synthetic promoter that contains the U3 region of a modified MoMuLV LTR with myeloproliferative sarcoma virus enhancer. Other promoter sequences are compatible with the AAV particles of the disclosure. In some embodiments, the promoter is a ubiquitous promoter. In some embodiments, the promoter is a tissue-specific promoter, such as an endothelial cell (EC)-specific promoter.

In some embodiments, the promoter is an inducible promoter. A polynucleotide sequence operatively linked to an inducible promoter may be configured to cause the polynucleotide sequence to be transcriptionally expressed or not transcriptionally expressed in response to addition or accumulation of an agent or in response to removal, degradation, or dilution of an agent. The agent may be a drug. The agent may be tetracycline or one of its derivatives, including, without limitation, doxycycline. In some cases, the inducible promoter is a tet-on promoter, a tet-off promoter, a chemically-regulated promoter, a physically-regulated promoter (i.e., a promoter that responds to presence or absence of light or to low or high temperature). This list of inducible promoters is non-limiting.

In some embodiments, the promoter comprises a CMV enhancer/promoter. In some embodiments, the sequence encoding the CMV promoter comprises or consists of the sequence of SEQ ID NO: 19, or a functional variant thereof, optionally having 80%, 90%, 95%, or 99% sequence identity thereto.

In some embodiments, the promoter comprises a CMV enhancer. In some embodiments, the CMV promoter comprises a CMV enhancer. In some embodiments, the sequence encoding the CMV enhancer comprises or consists of the sequence of SEQ ID NO: 6, or a functional variant thereof, optionally having 80%, 90%, 95%, or 99% sequence identity thereto.

In some embodiments, the promoter comprises a CMV promoter. In some embodiments, the CMV promoter comprises or consists of the sequence of SEQ ID NO: 7, or a functional variant thereof, optionally having 80%, 90%, 95%, or 99% sequence identity thereto.

In some embodiments, the polynucleotide lacks a promoter. In some embodiments, expression of an RNA from the viral genome may be driven by the 5′ ITR. In some embodiments, expression of a protein from the viral genome may be driven by an Internal Ribosome Entry Site (IRES).

### Transgene

As used herein, the term “transgene” refers to any genetic element that is operatively linked to a promoter. Transgenes include protein-coding sequences, RNA-coding sequences (e.g. microRNA, gRNAs, or sgRNAs), and gene-editing systems (e.g. CRISPR/Cas systems and the like). In some embodiments, the transgene comprises a sequence encoding any of the proteinases listed in Table 1. In some embodiments, the transgene comprises or consists of the sequence of encoding any of the proteinases listed in Table 1, or a functional variant thereof, or having 80%, 90%, 95%, or 99% sequence identity thereto.

TABLE 1

| | Common | Illustrative | NCBI |
|---|---|---|---|
| Proteinase | name | Substrates | Gene ID |
| MMP-1 | Collagenase-1 | Collagens I, II, III, | 4312 |
| | | VII, VIII & X, gelatin, | |
| | | aggrecan, versican, | |
| | | tenascin, MMP-2, -9, | |
| | | pro-TNFα, IL-βI, α1-PI. | |
| MMP-2 | Gelatinase-A | Collagens I, IV, V, VII, X, | 4313 |
| | | XI & XIV, gelatin, elastin, | |
| | | fibronectin, aggrecan, | |
| | | decorin, laminin 1 & 5, | |
| | | HA'ase-treated versican, | |
| | | galectin-3, MMP-1, -9, -13, | |
| | | α1-PI. | |
| MMP-9 | Gelatinase-B | Collagens IV, V, VII, X | 4318 |
| | | & XIV, gelatin, elastin, | |
| | | galectin-3, HA'ase- | |
| | | treated versican, | |
| | | fibronectin, IL-βI, α1-PI. | |
| MMP-10 | Stromelysin-2 | Collagens III, IV & V, | 4319 |
| | | gelatin, elastin, MMP-1, -8. | |
| MMP-11 | Stromelysin-3 | Casein, laminin, | 4320 |
| | | fibronectin, gelatin, | |
| | | collagen IV, α1-PI. | |
| MMP-12 | Metalloelastase | Collagen IV, elastin, | 4321 |
| | | gelatin, casein, laminin, | |
| | | fibronectin, vitronectin, | |
| | | entactin, α1-PI, fibrinogen, | |
| | | fibrin. | |
| MMP-13 | Collagenase-3 | Collagens I, II, III, IV, | 4322 |
| | | IX, X & XIV, gelatin, | |
| | | aggrecan, perlecan, | |
| | | tenascin C, fibronectin, | |
| | | osteonectin, MMP9. | |
| MMP-19 | RASI | Gelatin. | 4327 |
| ADAM-9 | Meltrin gamma | Ectodomain shedding, | 8754 |
| | | collagen XVII, pro-TNFα, | |
| | | heparin binding EGF-like | |
| | | growth factor, TNF | |
| | | receptor II. | |
| ADAM-12 | Meltrin alpha | Ectodomain shedding, | 8038 |
| | | heparin-binding EGF- | |
| | | like growth factor. | |
| ADAMTS-1 | Aggrecanase-3 | Aggrecan, versican VI. | 9510 |
| ADAMTS-4 | Aggrecanase-1 | Aggrecan, brevican, | 9507 |
| | | versican VI, fibromodulin, | |
| | | decorin. | |
| ADAMTS-5 | Aggrecanase-2 | Aggrecan, brevican, | 11096 |
| | | decorin, biglycan. | |
| Tissue PA | tPA | Plasminogen activation | 5327 |
| Urokinase PA | uPA | Plasminogen activation | 5328 |

In some embodiments, the transgene comprises a sequence encoding a matrix metalloproteinase 3 (MMP-3).

In some embodiments, the disclosure provides a unit dose comprising a plurality of recombinant adeno-associated virus of serotype 9 (rAAV9) particles, wherein each rAAV9 of the plurality of rAAV9 particles is non-replicating, and wherein each rAAV9 of the plurality of rAAV9 particles comprises a polynucleotide comprising, from 5′ to 3′ (a) a sequence encoding a 5′ inverted terminal repeat (ITR); (b) a sequence encoding a promoter; (c) a sequence encoding a matrix metalloproteinase 3 (MMP-3); (d) a sequence encoding a polyadenylation (polyA) signal; and (e) a sequence encoding a 3′ ITR.

In some embodiments, the sequence encoding MMP-3 comprises or consists of a nucleotide sequence encoding the MMP-3 amino acid sequence of SEQ ID NO: 1 or SEQ ID NO: 22, or a functional variant or functional fragment thereof, optionally having 80%, 90%, 95%, or 99% sequence identity thereto. In some embodiments, the nucleotide sequence encoding the MMP-3 amino acid sequence comprises a wild-type nucleotide sequence. In some embodiments, the sequence encoding MMP-3 comprises or consists of the nucleotide sequence of SEQ ID NO: 9, or sequence having 80%, 90%, 95%, or 99% sequence identity thereto.

In some embodiments, the sequence encoding MMP-3 is codon optimized. In some embodiments, the sequence encoding MMP-3 comprises or consists of the nucleotide sequence of SEQ ID NO: 23, or sequence having 80%, 90%, 95%, or 99% sequence identity thereto. In some embodiments, the sequence encoding MMP-3 comprises or consists of the nucleotide sequence of SEQ ID NO: 24, or sequence having 80%, 90%, 95%, or 99% sequence identity thereto. In some embodiments, the sequence encoding MMP-3 comprises or consists of the nucleotide sequence of SEQ ID NO: 25, or sequence having 80%, 90%, 95%, or 99% sequence identity thereto.

In some embodiments, the sequence encoding MMP-3 is CpG depleted. In some embodiments, the sequence encoding MMP-3 comprises or consists of the nucleotide sequence of SEQ ID NO: 26, or a sequence having 80%, 90%, 95%, or 99% sequence identity thereto. In some embodiments, the sequence encoding MMP-3 comprises or consists of the nucleotide sequence of SEQ ID NO: 27, or a sequence having 80%, 90%, 95%, or 99% sequence identity thereto.

### Other Vector Elements

In some embodiments, the polynucleotide comprises a sequence encoding a polyadenylation (polyA) signal. In some embodiments, polyA signal comprises a human growth hormone (hGH) polyA sequence. In some embodiments, the hGH polyA sequence comprises SEQ ID NO: 11 or SEQ ID NO: 20, or a sequence having 80%, 90%, 95%, or 99% sequence identity thereto. In some embodiments, the polyA signal comprises a bovine growth hormone (bGH) polyA signal or a rabbit β-globin polyA signal.

In some embodiments, the polynucleotide comprises an intron, e.g. a human β-globin intron such as SEQ ID NO: 8, or a sequence having 80%, 90%, 95%, or 99% sequence identity thereto. In some embodiments, the polynucleotide comprises a Kozak sequence, e.g. SEQ ID NO: 21.

In some embodiments, the polynucleotide comprises or consists of the sequence of SEQ ID NO: 3 or SEQ ID NO: 4, or a sequence having 80%, 90%, 95%, or 99% sequence identity thereto.

### Pharmaceutical Compositions

In some embodiments, the unit dose is sterile. In some embodiments, comprises a pharmaceutically acceptable carrier. Suitable carriers include, without limitation, physiological saline, saline with 100-200 mM sodium chloride, saline with 150 sodium chloride, saline containing a polyol (such as 5% sucrose), and the like. In some embodiments, the carrier comprises poloxamer, including without limitation poloxamer 188 or Pluronic F-68. Suitable concentrations of poloxamer include 0.0001%-0.01% or approximately 0.001%.

For purposes of injection, various solutions can be employed, such as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxypropyl cellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

### Recombinant Protein

In another aspect, the disclosure provides unit doses comprising recombinant matrix metalloproteinase 3 (MMP-3) protein. In some embodiments, the unit dose comprises between 0.1 nanograms (ng) and 10 ng, or between 0.5 ng and 5 ng, between 1 ng and 2 ng, between 3 ng and 4 ng, between 5 ng and 6 ng, between 7 ng and 8 ng, or between 9 ng and 10 ng, inclusive of the endpoints. In some embodiments, the unit dose is 0.1 ng or higher, 1 ng or higher, or 10 ng or higher. In another aspect, the unit dose is provided in a volume between 10 and 200 μl, or between 20-100 μl.

In some embodiments, the pharmaceutical dose comprises a concentration of the recombinant MMP-3 protein between 1 milligrams per milliliter (mg/ml) and 500 mg/mL, for example between 5 mg/mL and 200 mg/ml, between 10 mg/mL and 100 mg/ml, or between 20 mg/mL and 80 mg/ml.

In some embodiments, the unit dose comprises between 1 milligrams per milliliter (mg/mL) and 100 mg/mL, inclusive of the endpoints, of the recombinant MMP-3 protein. In some embodiments, the unit dose comprises between 1 mg/mL and 5 mg/mL, 5 mg/mL and 10 mg/mL, 15 mg/mL and 20 mg/mL, 20 mg/mL and 25 mg/mL, 25 mg/mL and 30 mg/mL, 30 mg/mL and 35 mg/mL, 35 mg/mL and 40 mg/mL, 40 mg/mL and 45 mg/mL, or 45 mg/mL and 50 mg/mL. In some embodiments, the unit dose comprises between 50 mg/mL and 55 mg/mL, 55 mg/mL and 60 mg/mL, 65 mg/mL and 70 mg/mL, 70 mg/mL and 75 mg/mL, 75 mg/mL and 80 mg/mL, 80 mg/mL and 85 mg/mL, 85 mg/mL and 90 mg/mL, 90 mg/mL and 95 mg/mL, or 95 mg/mL and 100 mg/mL.

In some embodiments, the unit dose comprises about 1 mg/mL, about 5 mg/mL, about 15 mg/mL, about 20 mg/mL, about 25 mg/mL, about 30 mg/mL, about 35 mg/mL, about 40 mg/mL, or about 45 mg/mL. In some embodiments, the unit dose comprises about 50 mg/mL, about 55 mg/mL, about 65 mg/mL, about 70 mg/mL, about 75 mg/mL, about 80 mg/mL, about 85 mg/mL, about 90 mg/mL, about 95 mg/mL, or about 100 mg/mL. In some embodiments, the unit dose comprises about 10 mg/mL of the recombinant MMP-3 protein.

In some embodiments, the recombinant MMP-3 protein is a human MMP-3 protein. In some embodiments, the recombinant MMP-3 protein has a polypeptide sequence that comprises or consist of the sequence of SEQ ID NO: 1, SEQ ID NO: 2, SEQ ID NO: 10 or SEQ ID NO: 22, or a functional variant or functional fragment thereof, optionally having 80%, 90%, 95%, 96%, 97%, 98%, or 99% sequence identity thereto.

### Methods

In another aspect, the disclosure provides methods of transducing the corneal endothelium of a subject. The methods comprise administering an effective amount of a unit dose as described herein. In some embodiments, the subject is a primate (e.g., a monkey, ape, or human). The subject may be male or female. The subject may be a juvenile or an adult. In some embodiments, the subject suffers from or is at risk for elevated intraocular pressure (IOP). In some embodiments, the subject suffers from or is at risk for elevated IOP due to a congenital disorder such as primary congenital glaucoma juvenile primary open angle glaucoma, MYOC glaucoma, and the like. In some embodiments, the subject suffers from or is at risk for elevated IOP due to advanced age. In some embodiments the subject has elevated IOP that has not yet advanced to glaucoma. In some cases, the subject has elevated IOP and glaucoma. In some cases, the subject has glaucoma without elevated IOP.

Administration of an effective dose of the compositions may be by routes standard in the art including, but not limited to, intracameral inoculation, intravitreal inoculation, subretinal inoculation, suprachoroidal inoculation, canaloplasty, or episcleral vein-mediated delivery. In an embodiment, the effective dose is delivered intracamerally.

As used herein, the term “patient in need” or “subject in need” refers to a patient or subject at risk of, or suffering from, a disease, disorder or condition that is amenable to treatment or amelioration with a rAAV comprising a nucleic acid sequence encoding matrix metalloproteinase or a composition comprising such a rAAV provided herein. A patient or subject in need may, for instance, be a patient or subject diagnosed with a disease associated with the malfunction of matrix metalloproteinase, such as ocular hypertension and/or glaucoma. A subject may have a mutation or a malfunction in a matrix metalloproteinase gene or protein. “Subject” and “patient” are used interchangeably herein.

The subject treated by the methods described herein may be a mammal. In some cases, a subject is a human, a non-human primate, a pig, a horse, a cow, a dog, a cat, a rabbit, a mouse or a rat. A subject may be a human female or a human male. Subjects may range in age, including juvenile onset glaucoma, early onset adult glaucoma, or age-related glaucoma. Thus, the present disclosure contemplates administering any of the rAAV vectors disclosure to a subject suffering from juvenile onset glaucoma, to a subject suffering from early onset adult glaucoma, or to a subject suffering from age-related glaucoma.

Combination therapies are also contemplated by the invention. Combination as used herein includes simultaneous treatment or sequential treatment. Combinations of methods of the invention with standard medical treatments (e.g., corticosteroids or topical pressure reducing medications) are specifically contemplated, as are combinations with novel therapies. In some cases, a subject may be treated with a steroid to prevent or to reduce an immune response to administration of a rAAV described herein. In certain cases, a subject may receive topical pressure reducing medications such as prostaglandin analogs, beta blockers, and/or ROCK inhibitors, before, during, or after administrating of an rAAV described herein. In certain cases, a subject may receive a medication capable of causing the pupil of the eye to dilate (e.g., tropicamide and/or phenylephrine). In certain cases, the subject may receive a moisturizing gel during recovery to prevent corneal dehydration. In some embodiments, the prostaglandin analog is latanaprost or bimatoprost. In some embodiments, the beta blocker is timolol. In some embodiments, the ROCK inhibitor is Rhopressa.

In some embodiments, transducing the corneal endothelium results in transduction of at least 15%, 20%, 25%, 30%, 35%, 40%, 45%, 50%, 55%, 60%, 65%, or 70% of corneal endothelium cells. Stated differently, the transduction efficiency may be, in some cases, 15%, 20%, 25%, 30%, 35%, 40%, 45%, 50%, 55%, 60%, 65%, 70%, or greater. As used herein, “transduction efficiency” refers to the ability of a vector (e.g. an AAV vector or AAV particle) to deliver a polynucleotide into a cell (e.g. a corneal endothelium cell). Transduction efficiency may be determined in vivo, such as using an AAV particle encoding a fluorescent marker (e.g. GFP or eGFP). Transduction efficiency may also be determined by immunohistochemical analysis of a tissue same. For example, sections of cornea samples from a treated subject may be stained using an anti-MMP3 antibody. Transduction efficiency is generally described as a fraction or percentage of target cells that receive a target polynucleotide. In the case of corneal endothelium, the target cells may be identified morphologically. Morphological identification of corneal endothelium cells may be performed using microscopy.

Transduction efficiency may be assessed in in vivo using various methods known in the art, including but not limited to Color and Fluorescent Anterior Segment Photography, Optical Coherence Tomography, and Immunohistochemistry. Color and fluorescent anterior segment photography may be performed, e.g., using a Topcon TRC50EX retinal camera with Canon 6D digital imaging hardware and FUNDUS PHOTO NEW VISION Ophthalmic Imaging Software. Illustrative settings for the color photos include a shutter speed (Tv) of 1/25 sec, ISO of 400 and flash 18. Illustrative settings for monochromatic and color fluorescent images with exciter and barrier filters engaged are 480 nm exciter, 525 nm barrier filter, a flash setting of 200, Tv ⅕ sec, ISO 3200 and Flash 300.

Anterior segment OCT may be performed using a Heidelberg Spectralis OCT HRA or OCT Plus with eye tracking and HEYEX image capture and analysis software. Autofluorescence function of the SPECTRALIS may be used to obtain images of GFP expression in the anterior chamber.

Immunohistochemistry may be performed using various known methods. Transfection efficiency may be determined by counting cells positive for a marker protein (e.g. GFP) or therapeutic protein (e.g., MMP-3) under a confocal microscope.

Transduction efficiency may also be assessed in vivo by measuring the concentration of a secreted protein, such as MMP-3. First, ocular fluid, such as aqueous humor and/or vitreous humor, is withdrawn from the subject and are stored under appropriate conditions, such as frozen, until evaluation. Next, the ocular fluid is assayed to measure the amount of the secreted protein present, for example using an ELISA assay or Western blot. The amount of the secreted protein present is quantified by comparing the signal obtained in the ELISA assay to a standard curve, which measures the signal of a known protein standard.

In some embodiments, the methods of the disclosure comprise administering a unit dose comprising rAAV9 particles, wherein each rAAV9 of the plurality of rAAV9 particles in the unit dose is a single-stranded AAV (ssAAV).

In some embodiments, a volume of 10 μl to 200 μl is injected into the anterior chamber of the eye. In some embodiments, this is a volume of between 20 μl to 100 μl. More specifically, the injected volume could be about 50 μl, about 60 μl, about 70 μl, about 80 μl, about 90 μl, or about 100 μl. In some embodiments, a volume of aqueous humor is first removed from the subject's eye prior to injection using a needle. The removal of aqueous is sometimes called an aqueous tap or paracentesis.

In some embodiments, the disclosure provides a method of transducing the corneal endothelium of a subject, comprising administering an effective amount of a unit dose comprising a plurality of recombinant adeno-associated virus of serotype 9 (rAAV9) particles, wherein the subject is a primate; wherein each rAAV9 of the plurality of rAAV9 particles is non-replicating; wherein each rAAV9 of the plurality of rAAV9 particles is a single-stranded AAV (ssAAV); wherein each rAAV9 of the plurality of rAAV9 particles comprises a polynucleotide comprising, from 5′ to 3′: (a) a sequence encoding a 5′ inverted terminal repeat (ITR); (b) a sequence encoding a promoter (e.g., a CMV promoter); (c) a sequence encoding a matrix metalloproteinase 3 (MMP-3); (d) a sequence encoding a polyadenylation (polyA) signal; (e) a sequence encoding a 3′ ITR. In some embodiments, the unit dose comprises (i) between 1×10⁷ vector genomes (vg) and 5×10¹² vg, inclusive of the endpoints, of rAAV9 particles; or (ii) about 1×10⁹ vector genomes (vg) per milliliter (mL) to 5×10¹³ vg/mL of rAAV9 particles.

Alternatively or in addition to other methods of assaying for transduction, transduction of the corneal endothelium may be assessed by measuring the concentration of exogenous protein expressed. For example, in some embodiments, the methods described herein result in expression of MMP-3 in the aqueous humor (AH) of an eye of the subject at a measured concentration of between 0.01 ng/mL and about 10 ng/mL, inclusive of the endpoints.

In some embodiments, the concentration of MMP-3 in the AH is between about 0.01 ng/mL and about 0.1 ng/mL, between about 0.1 ng/mL and about 1 ng/mL, between about 1 ng/mL and about 2 ng/mL, between about 2 ng/mL and about 4 ng/mL, between about 4 ng/mL and about 6 ng/mL, between about 6 ng/mL and about 8 ng/mL, between about 6 ng/mL and about 10 ng/mL, or between about 10 ng/mL and about 50 ng/mL, or greater.

In some embodiments, the concentration of MMP-3 in the AH is about 0.01 ng/mL, about 0.1 ng/mL, about 1 ng/mL, about 2 ng/mL, about 4 ng/mL, about 6 ng/mL, about 6 ng/mL, about 10 ng/mL, or about 50 ng/mL, or greater. In some embodiments, the concentration of MMP-3 in the AH is greater than 1 ng/mL. In some embodiments, the concentration is in the range of 1 ng/mL to 10 ng/mL.

The concentrations of MMP-3 (or another exogenous protein) in the AH may be measured by enzyme-linked immunosorbent assay (ELISA) or Western blot. Antibodies against MMP-3 useful in measuring the concentration in AH include those available from PROTEINTECH (17873-1-AP), ABCAM (ab53015), and R&D SYSTEMS (DMP300).

In some embodiments, the measured concentration of MMP-3 in the AH is greater than or equal to 1 ng/mL. In some embodiments, the measured concentration of MMP-3 in the AH is less than or equal to 10 ng/mL. In some embodiments, the measured concentration of MMP-3 in the AH is 1-10 ng/mL, inclusive of the endpoints. In some embodiments, the measured concentration of MMP-3 in the AH is at least 1-3 ng/mL, inclusive of the endpoints. In some embodiments, the concentration of MMP-3 is measured using radiolabeled MMP-3.

In some embodiments, the AAV particles and methods of the disclosure generated a sustained or prolonged expression of MMP-3 (or another transgene). In some embodiments, the expression of MMP-3 is maintained at least 21 days, 42 days, 56 days, or 66 days. In some embodiments, the expression of the expression of MMP-3 is maintained at least 90 days. In some embodiments, the expression of a transgene and/or an exogenous protein is maintained at least 21 days, 42 days, 56 days, or 66 days. In some embodiments, the expression of a transgene and/or an exogenous protein is maintained at least 90 days.

The present disclosure further relates to assessment of efficacy and safety of gene therapy vectors in in vitro assay systems. The disclosure provides a recombinant AAV (rAAV) vector comprising a polynucleotide sequence encoding matrix metalloproteinase 3 (MMP-3). Using this rAAV vector or vectors delivering transgene for other therapeutic proteins, one can treat vision conditions such as glaucoma by administering the rAAV to the eye. In some cases, treatments aim to lower ocular pressure, and one means of achieving lower ocular pressure is through remodeling or degrading the extracellular matrix by the therapeutic protein, such as MMP-3 or the like. The effect can be assessed by measuring the permeability of the extracellular matrix of the trabecular meshwork of the eye or by measuring in an in vivo assay the effect of the rAAV. Suitable in vitro assays disclosed by the present inventions include use of human Schlemm's canal (SC) endothelial cells (SCEC) monolayers derived from either human glaucomatous, primary open angle glaucoma (POAG) or control (cataract) cultured in aqueous humor (AH). Transendothelial electrical resistance (TEER) and permeability to a fluorescent-linked dye can then be measured in cells transduced with rAAV vector or not transduced for comparison. In other assays, ECM proteins can be stained and observed by immunofluorescence. These and other in vitro assays are described in more detail as follows.

Contacting the rAAV vector to a human trabecular meshwork (HTM) monolayer may increase the rate of tracer molecule flux through such a monolayer by more than about 5, 6, 7, 8, 9, 10, 11, 12, 13, or 15% over the tracer molecule flux through a HTM monolayer not contacted with said rAAV. As used herein, the terms “tracer molecule flux” or “tracer flux” refer to the flow of a tracer molecule across an epithelial membrane as described, for example, in Dawson et al. Tracer flux ratios: a phenomenological approach. J. Membr Biol. 31:351-58 (1997). Optionally, the tracer may be dextran conjugated to fluorescein isothiocyanate (FITC-dextran). In cases, contacting said rAAV vector to a human trabecular meshwork (HTM) monolayer decreases the transendothelial electrical resistance (TEER) of said monolayers by more than about 1, 2, 3, 4, 5, 6, 7, 8, 9, 10, 11, 12, 13, 14, or 15 Ohm per cm², more than about 15 Ohm per cm², or more than about 20 Ohm per cm² over the TEER of a monolayer not contacted with said rAAV. Methods of determining TEER are described in Srinivasan et al. TEER measurement techniques for in vitro barrier model systems. J. Lab Autom. 20:107-26 (2015).

In the eye of a subject, in vivo, administering the rAAV to the eye may, in some cases, increase permeability of the extracellular matrix of the trabecular meshwork, decrease outflow resistance of said eye, and/or decrease intraocular pressure (IOP). Measurement of outflow resistance and intraocular pressure of an eye is described in the examples that follow this detailed description, and in, for example, in Sherwood at al. (2016) Measurement of Outflow Facility Using iPerfusion. PLoS One, 11, e0150694. The methods of the disclosure, in some cases, increase the outflow facility of the treated eye by at least 25% or by at least 30%.

As used herein, “outflow facility” refers to the ratio of outflow rate to relevant pressure and is the reciprocal of hydrodynamic resistance. The commonly used approach for measuring outflow facility is based on mass conservation of the flow entering and exiting the eye during an in vivo perfusion according to:

Qᵢₙ+Q=C(P−Pₑ)+Q₀

which is known as the modified Goldmann equation. Qᵢₙ is the rate of AH secretion, Q is the flow rate into the eye from the perfusion apparatus and Q₀ is the pressure-independent outflow. P is the intraocular pressure and Pₑ is the pressure in the episcleral vessels (into which the AH drains). In this form, C is the total outflow facility, comprising both conventional outflow and any pressure-dependent components of unconventional outflow and AH secretion (pseudofacility). Herein we use the term “facility” to indicate C, for simplicity. In order to calculate facility, Q₀ and Qᵢₙ, Pₑ and C itself are often assumed to be pressure independent (thereby tacitly assuming a linear Q−P relationship).

Under these assumptions, two measurements of P and Q are thus sufficient to estimate the facility according to the two-step perfusion protocol:

$${C}_{lin} = \frac{{Q}_{II} - {Q}_{I}}{{P}_{II} - {P}_{I}} \qquad Equation 1$$

where the subscripts I and II denote measurements at two different pressures, and Cₗᵢₙ is a pressure independent facility, based on the assumption of a linear Q−P response. Alternatively, for the case of enucleated eyes, Qᵢₙ and Pₑ are zero, hence Eq 1 reduces to:

Q=CₗᵢₙP+Q₀ Equation 2

In order to provide a more robust method, it is possible to measure multiple (two to ten) points and fit a power-law model to the Q−P relationship to capture the pressure dependence of outflow facility

$$C ( P ) = {{C}_{r} ( \frac{P}{{P}_{r}} )}^{β} \qquad Equation 3$$

Where Pᵣ is a reference pressure defined to be 8 mmHg in enucleated mouse eyes, at which Cᵣ is the facility. The power exponent β characterizes the non-linearity of the flow-pressure relationship and can be interpreted as an index of the combined sources of non-linearity affecting the flow-pressure relationship through the outflow pathway. Additional refinements in primate in vivo perfusions include the introduction of three stepping cycles with a spontaneous IOP reading before and after each cycle to account for temporal and pressure dependent responses.

The intraocular pressure (IOP) of a subject or a mammal to which a composition is administered may be decreased by more than 1, 2, 3, 4, 5, 6, 7, 8, 9, or 10 mmHg. The outflow rate may be increased by 0.1-0.5 μL/min/mmHg. The outflow rate may be increased by more than 0.1, 0.2, 0.3, 0.4, or 0.5 μL/min/mmHg. The outflow rate may be increased by more than 1, 2, 3, 4, 5, 10, or 15 μL/min/mmHg, or more than 20%, 30%, 40%, or 50%. The optically empty length in the trabecular meshwork of a subject or mammal may be increased by more than about 5, 10, 15, 20, 25, 30, 35, 40, 45, or 50%. Generally, rAAV vectors cause transduction of cells to which they are contacted. The transduced cells may be cells of the corneal endothelium, as well as other ocular cells. After administration, MMP-3 concentration in aqueous humor of may increase by about 0.1, 0.2, 0.3, 0.4, 0.5, or 0.6 ng/ml, or any value in between, such as in particular an increase of about 1 ng/ml or greater. In some embodiments, the MMP-3 concentration may be between about 0.1 to about 10 ng/ml. In some embodiments, the MMP-3 concentration may be between about about 1 to about 10 ng/ml. In some embodiments, the MMP-3 concentration may be between about 1 to about 5 ng/ml. In some embodiments, the MMP-3 concentration may be between about 1 to about 2 ng/ml. In some embodiments, MMP-3 activity in aqueous humor of said eye is increased by about 1, 2, 3, 4, 5, or 6, mU or greater, or any value in between, such as in particular by about 5.34 mU or greater. It is further disclosed that the corneal thickness of said mammal is unchanged following treatment. In some embodiments, the decrease in IOP and/or increase in outflow facility occurs within about 30 days, about 40 days, about 50 days, about 60 days, about 70 days, or about 80 days of the administering step. In some embodiments, the decrease in IOP and/or increase in outflow facility occurs within about 66 days of the administering step.

In some embodiments, the corneal thickness remains unchanged relative to corneal thickness in the subject before the administering step and/or relative to corneal thickness in a subject administered a control unit dose. As used here, “corneal thickness” refers to the distance between the outer boundaries of the corneal epithelium and corneal endothelium. Corneal thickness may be determined by corneal pachymetry. Corneal pachymetry may be performed, e.g., using an ACCUTOME ACCUPACH 5 ultrasound pachymeter or the equivalent. A mean pachymetry measure, in microns, is generally obtained from a series of four or more successive measures in each eye.

Alternatively or in addition to pachymetry, corneal thickness may be assessed by specular microscopy. Specular microscopy may be performed, e.g., with a TOMEY EM-3000 Specular Microscope or the like. Specular microscopy also may be used to evaluate integrity of the corneal endothelium.

In some embodiments, the methods of the disclosure cause no inflammatory response, or an inflammatory response that is not clinically significant. Methods of assessing inflammation of the cornea include slit lamp biomicroscopy. Anterior chamber cells, aqueous flare, and other ophthalmic findings may be graded using a modified Hackett-McDonald scoring system and composite clinical Scores derived from the sum of individual components of the score determined. See McDonald, T. O., and Shadduck, J. A. Eye irritation. Advances in Modern Toxicology. 139-191 (1977). Hackett, R. B., and McDonald, T. O. Assessing ocular irritation. Dermatotoxicology. 5th ed. 557-567 (1996).Slit lamp biomicroscopy and fundoscopy shows no evidence of intraocular inflammation over the course of the study (i.e. over at least 90 days)

In some embodiments, the method results in serum levels of MMP-3 that are not elevated over a baseline level of MMP-3 in the serum of the subject. No elevation of serum MMP3 demonstrates that there is no unbound MMP3 exiting the eye and entering the circulation. This is believed reduces the potential for off-target effects.

In another aspect, the disclosure provides methods of reducing intraocular pressure (IOP) in at least one eye of a subject, comprising administering an effective amount of any unit dose of the disclosure. In some embodiments, the subject is a mammal. In another embodiment, the subject is a primate.

In another aspect, the disclosure provides methods of treating and/or preventing elevated IOP and/or glaucoma in a subject in need thereof, comprising administering an effective amount of any unit dose of the disclosure. In some embodiments, the subject is a mammal. In other embodiments, the subject is a primate.

In any of the methods of the disclosure, improvement, reduction, increase, and/or prevention may be determined with reference to control subject not receiving the treatment, or with reference to a control eye. For example, treatment may be performed on one eye and the contralateral eye used as a control. Alternatively, or in addition, improvement, reduction, increase, and/or other changes may be determined in reference to a baseline measurement. As used herein, a “baseline” refers to a measurement, or an average of several measurements, taken before administration of the unit dose.

These embodiments and other embodiments may be further understood with reference to the Examples that follow.

### EXAMPLES

### Example 1: Recombinant Human MMP-3

This example characterizes the effect of intracameral delivery of recombinant human MMP3 (rhMMP3) on aqueous outflow and the morphology of the trabecular meshwork and Schlemm's canal of the African green monkey. The example demonstrates increasing outflow facility in a primate using MMP-3. The mean increase was about 30% with some subjects exhibiting an increase in outflow of at least 50%, 80%, 100%, 150%, 200%, or greater. The example further demonstrates that rhMMP3 has a dose dependent effect on aqueous outflow dynamics and intraocular pressure.

Recombinant human MMP-3 (rhMMP3) lacking the pro-peptide domain (SEQ ID NO: 2) was expressed in bacterial cells and purified using standard methods. The main cause of elevated IOP in primary open angle glaucoma (POAG) is thought to be an increased outflow resistance. Rocha-Sousa et al. ISRN Ophthalmol 2013:261386 (2013). Furthermore, the pressure lowering medications latanoprost and rhopressa are known to increase outflow and reduce IOP Significantly, the example establishes that increases in outflow facility, known to be correlated with decreasing or preventing elevated intraocular pressure (IOP), is achieved when the concentration of the MMP-3 in the aqueous humor of the eye exceeds about 1 nanograms per milliliter (ng/mL). This establishes a critical correlate for therapeutic efficacy of any treatment designed to treat or prevent elevated IOP and/or glaucoma.

Each monkey (n=17) received an intracameral infusion between 0.2 mL and 2 mL of rhMMP3 formulated in phosphate buffered saline (PBS) at 10 ng/mL, concentration. The rhMMP3 was allowed to infuse into the eye, at a pressure of 5 mmHg above spontaneous (or resting) IOP for a one hour “preconditioning” before facility measurement. Contralateral controls were infused with vehicle and outflow facility was determined after this one-hour preconditioning phase.

At designated time points (before and after one hour of preconditioning), 100 μl of aqueous humor was aseptically collected from both eyes using a 0.3 ml syringe with a 3-gauge needle and stored at −70° C. until analysis by enzyme linked immunosorbent assay (ELISA). Samples were run in duplicate according to standard protocols for the R&D SYSTEMS Human Total MMP-3 Quantikine ELISA Kit.

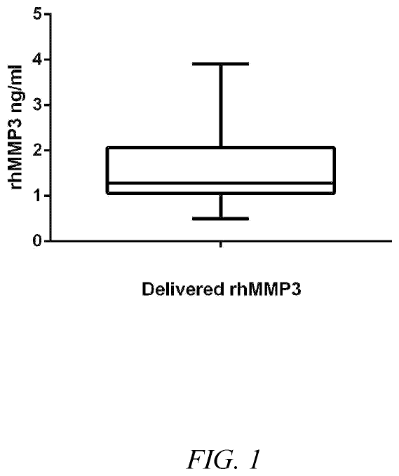

As shown in FIG. 1, recombinant human MMP3 was infused into the anterior chambers of primates in vivo. Infusion for 1 hour at 5 mmHg above spontaneous intraocular pressure resulted in a range of concentrations in the aqueous humor 0-4 ng/ml (or more specifically, 0.5-3.9 ng/ml).

Outflow facility was measured using a modified iPerfusion system (Sherwood et al. (2016) Measurement of Outflow Facility Using iPerfusion. PLoS One, 11, e0150694) to allow for outflow measurement in the primate eye in vivo.

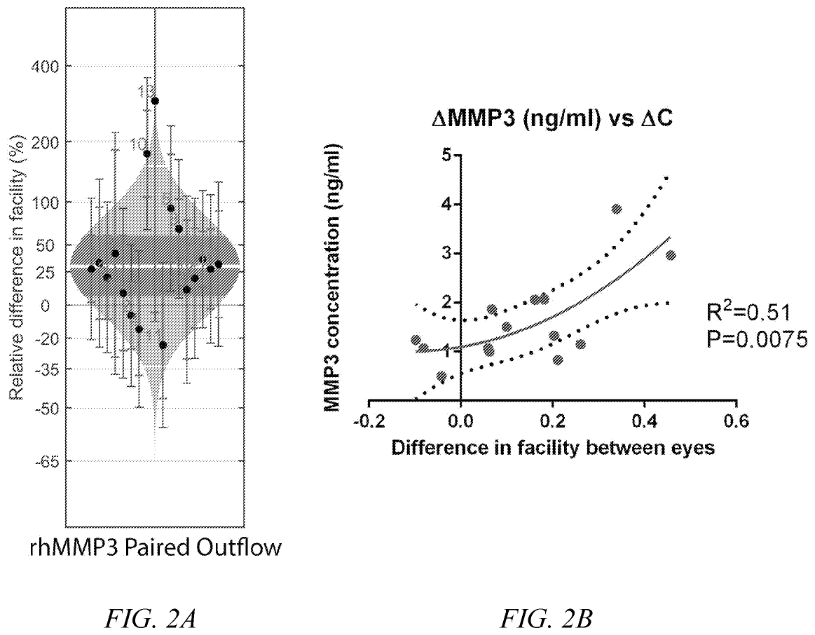

As shown in FIG. 2A, the difference in facility between each control and treated pair was mapped to the delivered dose of rhMMP3. RhMMP3 increases outflow facility in vivo by 30% in primates (P=0.017, N=17). Data points represent the percentage difference in facility between control and treated contralateral eyes. Dark shaded region represents the 95% confidence interval bounds of the white mean line. Error bars represent 95% confidence interval and 2 standard deviations.

FIG. 2B shows the amount of delivered rhMMP3 was correlated to the difference in outflow facility between eyes. On average, treated eyes exhibited an increase of 0.13 μl/min/mmHg; this varies with rhMMP3 concentration. Concentrations of MMP-3 in the aqueous humor were plotted against the relative difference in outflow facility for treated eye and control eye, resulting in a dose-response curve. The observed dose response was statistically significant (R²=0.51; p=0.0075). On average, treated eyes exhibited an increase of 0.13 μl/min/mmHg; this varies according to the concentration of rhMMP3 concentration.

### Example 2: Adeno-Associated Virus (AAV)

This example demonstrates transduction of the corneal endothelium of a primate using an AAV9 vector that expresses enhanced green fluorescence protein from a CMV promoter (AAV9-CMV-eGFP). The example demonstrates that transduction of the corneal endothelium may be achieved at AAV doses as low as 5×10¹¹ vg. The example further demonstrates unexpectedly superior transduction of the corneal endothelium of a primate using a single-stranded AAV vector (ssAAV9-EGFP) compared to a self-complementary AAV vector (scAAV9-EGFP).

Unit doses comprising AAV particles generated from each test vector (scAAV9-EGFP or ssAAV9-EGFP), as well as the control vectors, were prepared at 3.3×10¹³ vector genomes per milliliter (vg/mL) and 1×10¹³ vg/mL, respectively. The titer was measured using qPCR following incubations with DNase and proteinase K. Samples are diluted and run in quadruplicate using a master mix containing 2X TAQMAN Universal Master Mix, 20X TAQMAN Gene Expression Assay probes targeting the GFP or MMP-3 gene, and RNase-free water. Samples are compared against a standard curve of known concentration and reference standards. The qPCR reaction is performed on a STEPONEPLUS (Applied Biosystems®) instrument for 40 cycles of denaturing and annealing, with a prior 10 minute polymerase activation step. Data is analyzed on the qPCR instrument. Controls included 0.9% saline vehicle.

African green monkeys with normal slit lamp exams and fundus exams, color fundus photographs (CFP), and optical coherence tomography (OCT) were selected for the study. All procedures were performed under anesthesia: intramuscular ketamine (8 mg/kg) and xylazine (1.6 mg/kg), and pupil dilation with topical 10% phenyleprhine. Each subject received treatment in both eyes (OD=Oculus Dexter; OS=Oculus Sinister). A volume of 50 μL was administered by intracameral injection, resulting in a delivered dose of 5×10¹⁰ 1.65×10¹² vector genomes (vg) scAAV9 in the OD of each subject, and of 5×10¹¹ vg ssAAV9 in the OS of each subject. An eye speculum was placed in the eye to facilitate injections followed by a drop of proparacaine hydrochloride 0.5%, then 5% Betadine solution, and a sterile saline rinse. Intracameral injections were performed in both eyes (OU). Injections were performed using 31-gauge 0.5-inch long needle connected to 0.3-mL syringe. The needle was introduced through the temporal cornea approximately 2 mm anterior to the limbus without disturbing the intraocular structures. Following both intracameral injections, topical triple antibiotic neomycin, polymyxin, bacitracin ophthalmic ointment (or equivalent) was administered.

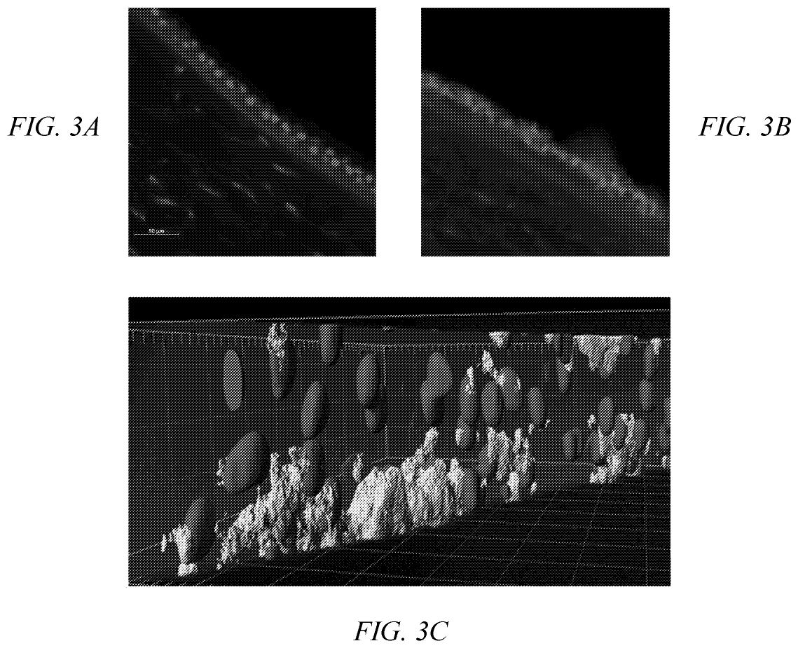

FIGS. 3A-3C show results for primate corneal transduction by AAV9. As shown in FIG. 3A, expression of the fluorescent eGFP marker in the corneal endothelium of primates treated by intracameral injection of self-complementary AAV9 (scAAV9-EGFP) was not distinguishable from negative controls at the sensitivity level of this assay. As shown in FIG. 3B, corneal endothelium from subjects intracamerally injected with single stranded AAV9 (ssAAV9-EGFP) demonstrated expression of the reporter gene, eGFP. Expression was restricted to the corneal endothelium. FIG. 3C shows a 3D rendering of Z-stacks from an eye injected with ssAAV9-eGFP (as in FIG. 3B). This rendering demonstrates the perinuclear expression of GFP in a large percentage of cells in the corneal endothelial layer.

Expression of the fluorescence reporter in the corneal endothelium continued for at least 90 days. At termination of the study more than 90 days after intracameral injection of the unit dose, GFP signal was observed by immunohistochemistry of from anterior chamber sections of the eyes of the subject primates.

### Example 3: Adeno-Associated Virus (AAV)

Example 1 established a target range for clinically effective expression of matrix metalloproteinase 3 (MMP-3). The range is at least about at least about 1 nanograms per milliliter (ng/mL), or between about 1 ng/mL and about 10 ng/mL, or between about 1 ng/mL and about 3 ng/mL. Due to the results of Example 2, a single-stranded AAV was selected.

### A. Expression of MMP-3 at Target Concentration Range in the Aqueous Humor

Part A demonstrates that transduction of the corneal endothelium of a primate using an AAV9 vector results in expression of matrix metalloproteinase 3 (MMP-3) and such therapeutically relevant levels—i.e., at least about 1 nanograms per milliliter (ng/mL). A single-stranded AAV9 vector expressing MMP-3 from a CMV promoter (AAV9-CMV-MMP3) was compared against a GFP control (AAV9-CMV-eGFP). Treatment assignment is shown in Table 4.

TABLE 4
Treatment Assignment

| Monkey | Treatment | Eye | Route | Titer (vg/ml) | Volume | Dose (vg) |
|---|---|---|---|---|---|---|
| 1 | AAV9-CMV- MMP3 | OD | intracameral | 1 × 10¹³ | 50 μL | 5 × 10¹¹ |
| | | OS | intracameral | 1 × 10¹³ | 50 μL | 5 × 10¹¹ |
| 2 | AAV9-CMV- MMP3 | OD | intracameral | 1 × 10¹³ | 50 μL | 5 × 10¹¹ |
| | AAV9-CMV- EGFP | OS | intracameral | N/A | 50 μL | N/A |

Imaging: Color and fluorescent anterior segment photography was performed using a Topcon TRC-50EX retinal camera with Canon 6D digital imaging hardware and New Vision Fundus Image Analysis System software. For the color photos the shutter speed (Tv) 1/25 sec, ISO 400 and Flash 18 were used. Monochromatic and color fluorescent images were acquired with exciter and barrier filters engaged (480 nm exciter/525 nm barrier filter), a flash setting of 200, Tv ⅕ sec, ISO 3200 and Flash 300. Fluorescence images were collected to serve as negative controls eyes receiving GFP vectors.

Optical Coherence Tomography: Anterior segment OCT was performed OU using a Heidelberg SPECTRALIS OCT HRA or OCT Plus with eye tracking and HEYEX image capture and analysis software. At the time of OCT measurement, the autofluorescence function of the SPECTRALIS was used to obtain images of GFP expression in the anterior chamber.

### B. Expression of MMP-3 in a Primate after AAV-Based Gene Therapy with MMP-3

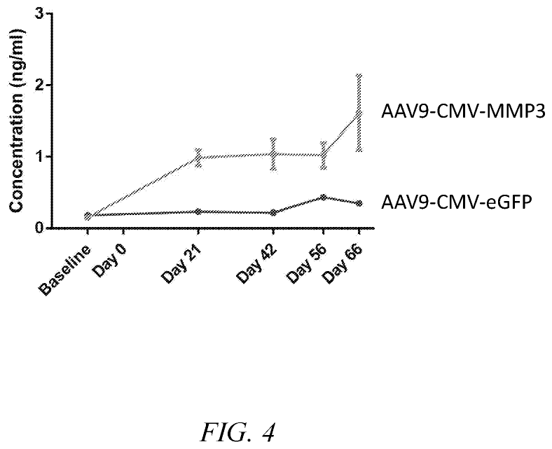

Part B demonstrates expression of MMP-3 at >1 ng/mL, which is sustained for at least 66 days. As shown in FIG. 4, intracameral injection with AAV9 expressing MMP3 (AAV9-CMV-MMP3) resulted in a concentration in the aqueous humor of the eye determined by ELISA in the range of about 1 ng/ml to 2 ng/ml over the selected time points (top line). One subject had a concentration in the range of 3-4 ng/ml. The time points at which the concentration of MMP-3 was assessed were days 21, 42, 56, and 66 after injection. The time points correspond to 3 weeks, 7 weeks, 8 weeks, or 9-10 weeks; or to 1 month or 2 months. Expression of MMP-3 in aqueous humor of eyes injected with AAV9-CMV-EGFP was not increased (bottom line).

Expression of MMP-3 in subjects treated with AAV9-CMV-MMP3 was an average of 1.6 ng/ml at the final time point. This was a significant increase compared to vehicle control (AAV9-eGFP) which remained at a concentration of <1 ng/ml for each time point. Individual subjects achieved an expression of MMP-3 of 3-4 ng/ml at the final time point.

### C. Reducing IOP in a Primate Using AAV-Based Gene Therapy with MMP-3

Part C demonstrates reducing IOP in a primate using AAV-based gene therapy with MMP-3. Part C further demonstrates a dose response relationship between expression of MMP-3 caused by the AAV-based gene therapy and effect on intraocular pressure (IOP).

At designated time points, intraocular pressure (IOP) was measured OU within ten minutes of sedation after placement of the monkey in a supine position. IOP measures were performed with a TONOVET tonometer set to the dog (d) calibration setting. Three measures were taken from each eye at each examination time point and the mean ITOP defined.

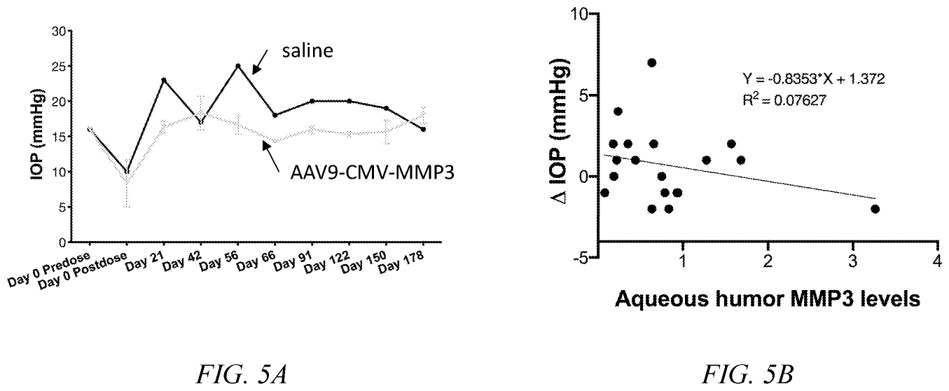

FIGS. 5A-5B show the treatment effect of AAV-MMP3 on intraocular pressure (IOP). FIG. 5A shows mean IOP±SEM (standard error of the mean) measured as mmHg. Measurements were taken at days 21, 42, 56, 66, 91, 122, 150, and 178 (corresponding to weeks 3, 6, 8, 9-10, 13, 17-18, 21-22, and 25-26; and corresponding to months 1, 2, 3, 4, 5, and 6). IOP measures remained stable beyond an immediate post-dose decrease in monkeys treated with AAV9-CMV-MMP3, with a decrease in IOP observed from about day 56 to about day 150. FIG. 5B shows reduced IOP with increasing levels of MMP3 in the aqueous humor measured 66 days after administration of the treatment.

As shown in FIG. 5A, apart from the expected reduction immediately post dose, IOP remained stable at evaluated time points in eyes treated with AAV9-CMV-MMP3. On average, treated eyes demonstrate a consistently reduced IOP over the course of the experiment post injection with AAV-MMP3. As shown in FIG. 5B, a comparison of MMP3 levels in aqueous humor versus change in IOP from baseline revealed a reduced IOP with increasing levels of MMP3.

### D. AAV-Based Gene Therapy with MMP-3 does not Impact Corneal Thickness

Part D demonstrates that AAV-based treatment with MMP-3 does not impact corneal thickness. Corneal thickness was measured by pachymetry and specular microscopy throughout the course of a safety study. Corneal thickness measures remained stable and within normal limits and no AAV-MMP3 associated changes were evident.

Specular microscopy: At designated time points, specular microscopy was performed with a TOMEY EM-3000 Specular Microscope to evaluate integrity of the corneal endothelium. The number of analyzed endothelial cells, cell density, average, standard deviation, coefficient of variation (CV) and range of cell dimensions were quantified.

Pachymetry: Corneal pachymetry was performed at designated time points using an ACCUTOME ACCUPACH 5 ultrasound pachymeter. A mean pachymetry measure, in microns, was obtained from a series of four successive measures in each eye.

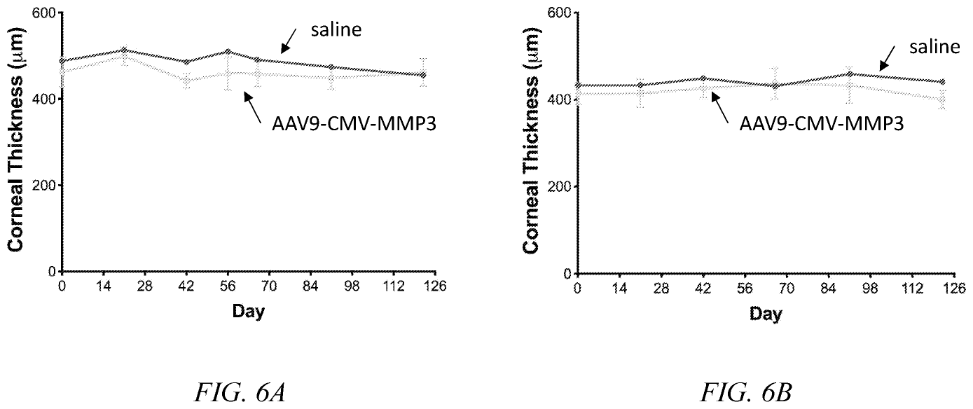

FIG. 6A-6B shows that corneal thickness measurements remained unchanged in response to AAV-MMP3. As shown in FIG. 6A, representative of mean corneal thickness measurements by pachymetry on a primate at the selected time points in response to AAV-MMP3 showed no significant changes over time. As shown in FIG. 6B, a representative of mean corneal thickness as measured by specular microscopy also demonstrates stable values over the course of the study, with no observed differences in thickness associated with AAV-MMP3.

### E. AAV-Based Gene Therapy with MMP-3 does not Cause Inflammation

Part E demonstrates that AAV-based treatment with MMP-3 causes no inflammatory response or a minimal inflammatory response. At designated time points, slit lamp biomicroscopy was be performed in both eyes (OU). Anterior chamber cells, aqueous flare, and other ophthalmic findings were graded using a modified Hackett-McDonald scoring system and composite Clinical Scores derived from the sum of individual components of the score were determined. Slit lamp biomicroscopy and fundoscopy shows no evidence of intraocular inflammation over the course of the study (i.e. over at least 90 days). In some animals, there was a minimal inflammatory response observed.

### F. AAV-Based Gene Therapy does not Elevate Serum Concentrations of MMP-3

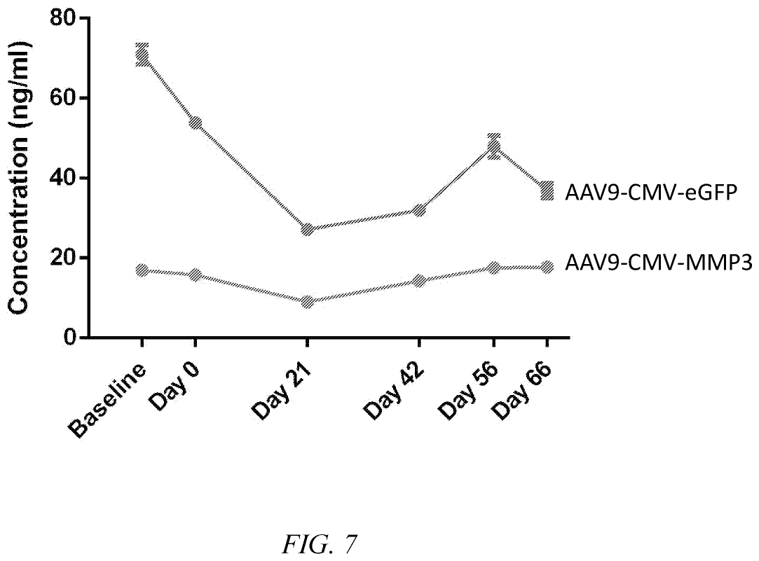

Part F demonstrates that AAV-based treatment with MMP-3 causes no increase in serum levels of MMP-3 over baseline. Blood (5 mL) was collected at designated phlebotomy time points after intracameral injection, for serum preparation by incubation in centrifuge tubes (without clot activators) for 1 hour at room temperature to allow clotting followed by centrifugation at 3000 rpm for 10 minutes at 4° C. ELISA was performed to measure total MMP3 concentration. No significant elevation was found at any time point in the AAV-MMP3-injected primate compared to baseline before injection. As shown in FIG. 7, MMP3 levels in serum (ng/mL) as determined by ELISA were not significantly elevated over baseline (bottom line). Levels were also not greater than those observed in a vehicle control (top line).

### Example 4: AAV9-Expressed MMP3 Reduces IOP and Increases Outflow Facility in a Murine Model of Steroid-Induced Glaucoma

### AAV9-Expressed MMP3 Reduces IOP in a Glucocorticoid Model of Ocular Hypertension

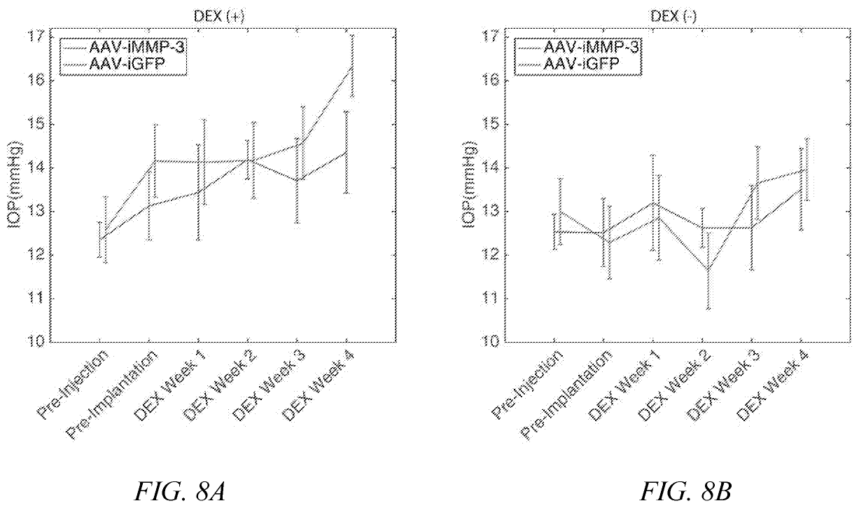

Wild-type mice were intracamerally injected with a tetracycline-inducible AAV encoding MMP3 (AAV-iMMP-3) or tetracycline-inducible AAV encoding GFP (AAV-iGFP) as control. Two weeks after injection of the AAV, mice were subcutaneously implanted with osmotic mini pumps, filled with the glucocorticoid dexamethasone. A control subset of mice were implanted with pumps secreting the vehicle control, cyclodextrin. In dexamethasone-treated animals, hypertension developed over the course of four weeks after implantation, as seen in FIG. 8A. Intraocular pressure was stable in cyclodextrin control-treated animals (FIG. 8B).

Two weeks after implantation, doxycycline (a tetracycline analog) was topically applied to the eye twice daily to induce transcription of the AAV. From the point of addition of doxycycline onward (DEX week 2), IOP in AAV-MMP3 treated eyes appears stable in hypertensive animals (FIG. 8A, bottom line) but continued to increase in AAV-iGFP animals (FIG. 8A, top line).

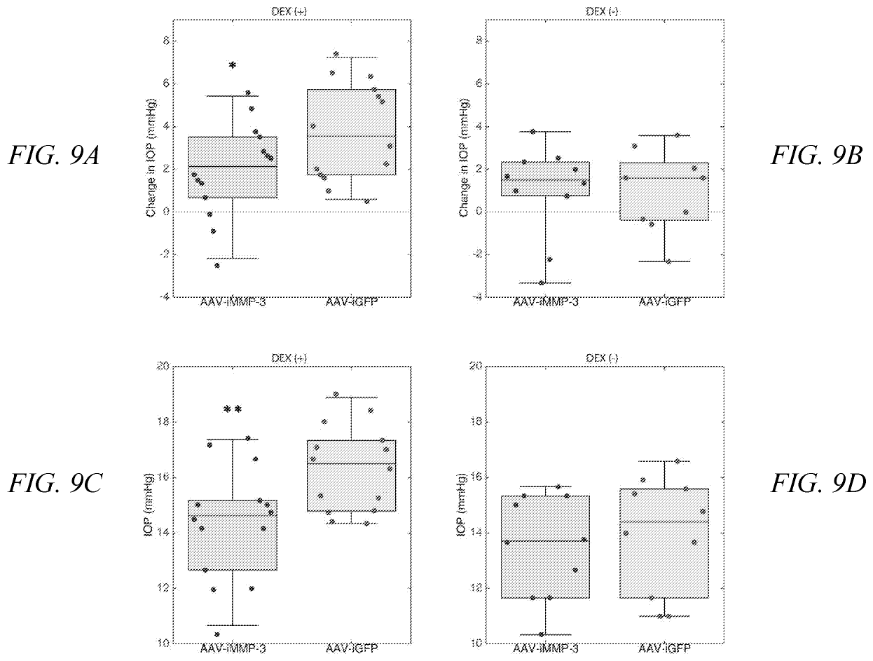

AAV-iMMP3 treated eyes have a significantly reduced IOP compared to AAV-iGFP controls when comparing the change in IOP from baseline to the final timepoint (6 weeks total, FIG. 9A). IOP is also significantly reduced in AAV-iMMP3 treated eyes in hypertensive animals when comparing the final timepoint only between contralateral eyes (FIG. 9C). No significant change is observed in normotensive control mice, FIG. 9B and FIG. 9D). Statistics were performed with Student's t-test. N=14 for dexamethasone-treated mice. N=10 for cyclodextrin-treated mice.

### AAV9-Expressed MMP3 Increases Outflow Facility in a Glucocorticoid Model of Ocular Hypertension

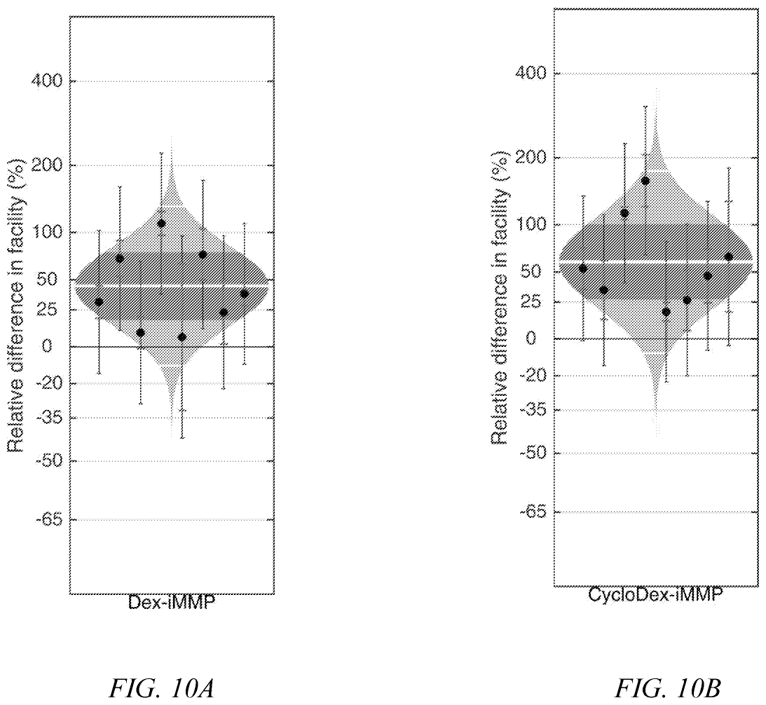

In both dexamethasone-induced ocular hypertensive (FIG. 10A) and normotensive mice FIG. 10B), outflow facility is increased by approximately 50% in AAV-MMP3 treated eyes compared to contralateral controls. In hypertensive animals, the average increase in outflow facility was 45% [18, 78] (mean percentage, [lower confidence interval, upper confidence interval]), P=0.0049, N=8. In normotensive animal controls, the average increase in outflow facility was 59% [26, 100], P=0.002, N=8.

### AAV-MMP3 Induces Extracellular Degradation at the Conventional Outflow Tissue in Mouse Models of Glucocorticoid Induced Ocular Hypertension

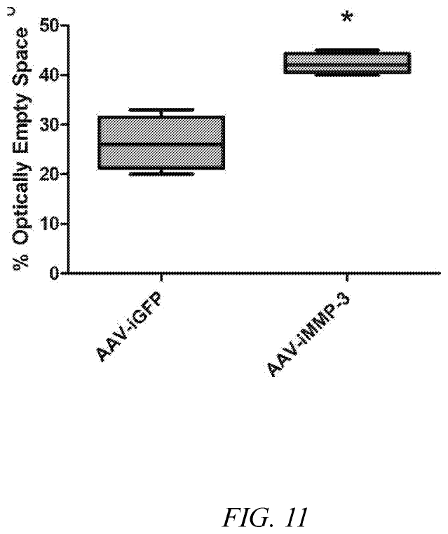

The subendothelial region of the Schlemm's Canal was quantified for the absence of extracellular matrix (ECM) material in both treated and control eyes using electron microscopy (FIG. 11). Treated eyes had a significantly greater amount of visually empty spaces at this region around the entire circumference of the eye.

### Example 5: AAV9-Expressed MMP3 Reduces IOP and Increases Outflow Facility in a Murine Model of Congential Glaucoma

### AAV9-Expressed MMP3 Reduces IOP in a Genetic Model of Glaucoma

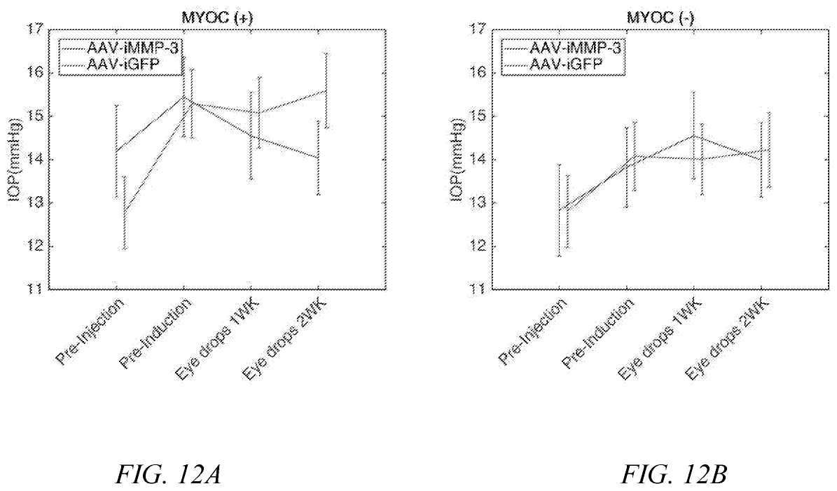

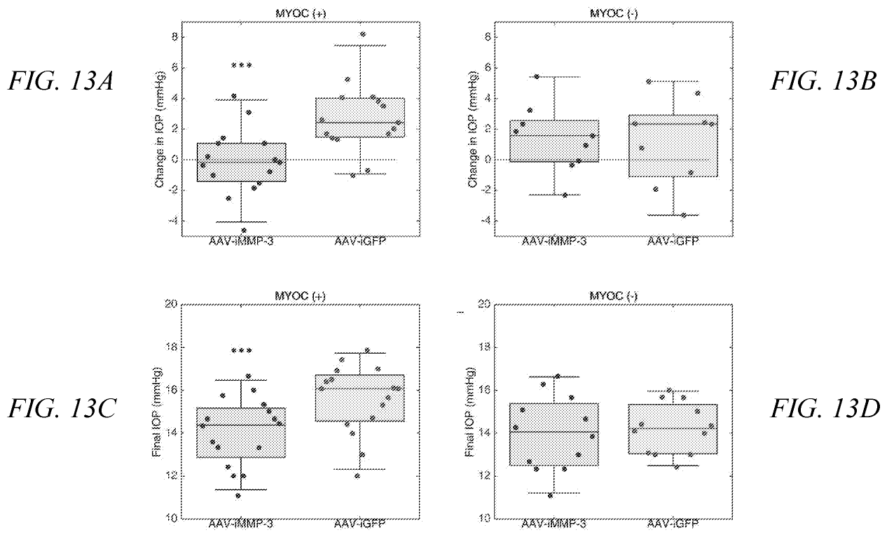

Wild-type mice (MYOC (−) in figures) and mice positive for the human mutant myocilin Y437H transgene (MYOC (+) in figures) were injected with AAV-iMMP3 in one eye and with AAV-iGFP as a contralateral control. Two weeks later, expression was induced via the twice daily administration of doxycycline eye drops. FIG. 12A shows that in hypertensive MYOC(+) animals, AAV-iMMP3-treated eyes exhibit a decrease in IOP over the course of the study. The median change in IOP of AAV-iMMP-3 and AAV-iGFP contralaterally treated eyes over the course of the experiment is presented in dot-box plots for the MYOC (+) group (FIG. 13A) and the MYOC (−) group (FIG. 13B). The final IOP readings are presented in (FIG. 13C) and (FIG. 13D) corresponding to MYOC (+) and MYOC (−) groups respectively. Difference in final IOP reading between contralateral eyes were significant in MYOC (+) animals (1.7±0.1 mmHg, p=0.0003, n=16, FIG. 6C) but not in MYOC (−) animals (0.1±0.2 mmHg, p=0.48, n=12, FIG. 6D).

### AAV9-Expressed MMP3 Increases Outflow Facility in a Genetic Model of Glaucoma

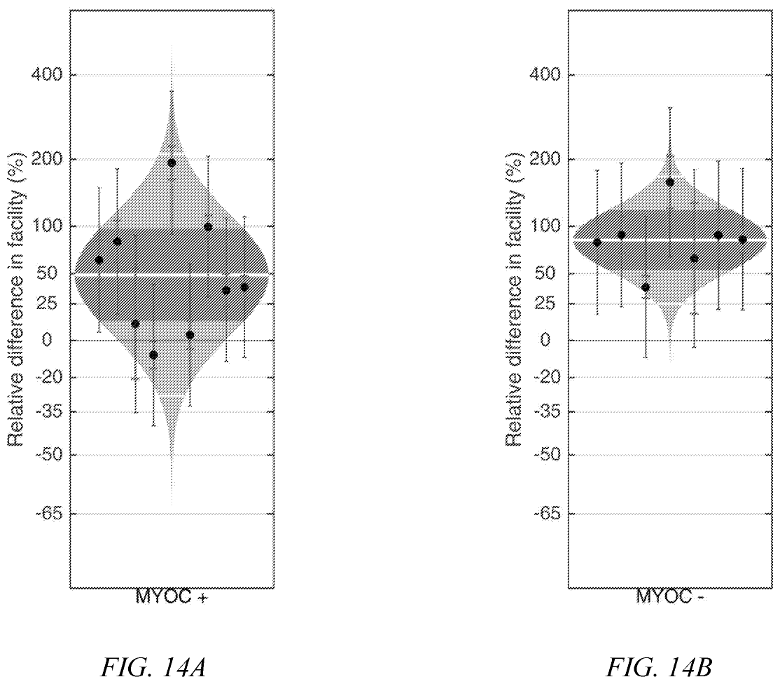

In MYOC^{Y437H} mice (FIG. 14A), outflow facility is increased by an average of 49%, P=0.0115, N=9. In normotensive littermate controls (FIG. 14B), the average increase in outflow facility was 88%, P=0.0001, N=8.

### Example 6: Development and Characterization of Codon-Optimized MMP3 Sequences

### Development of Codon Optimized Sequences

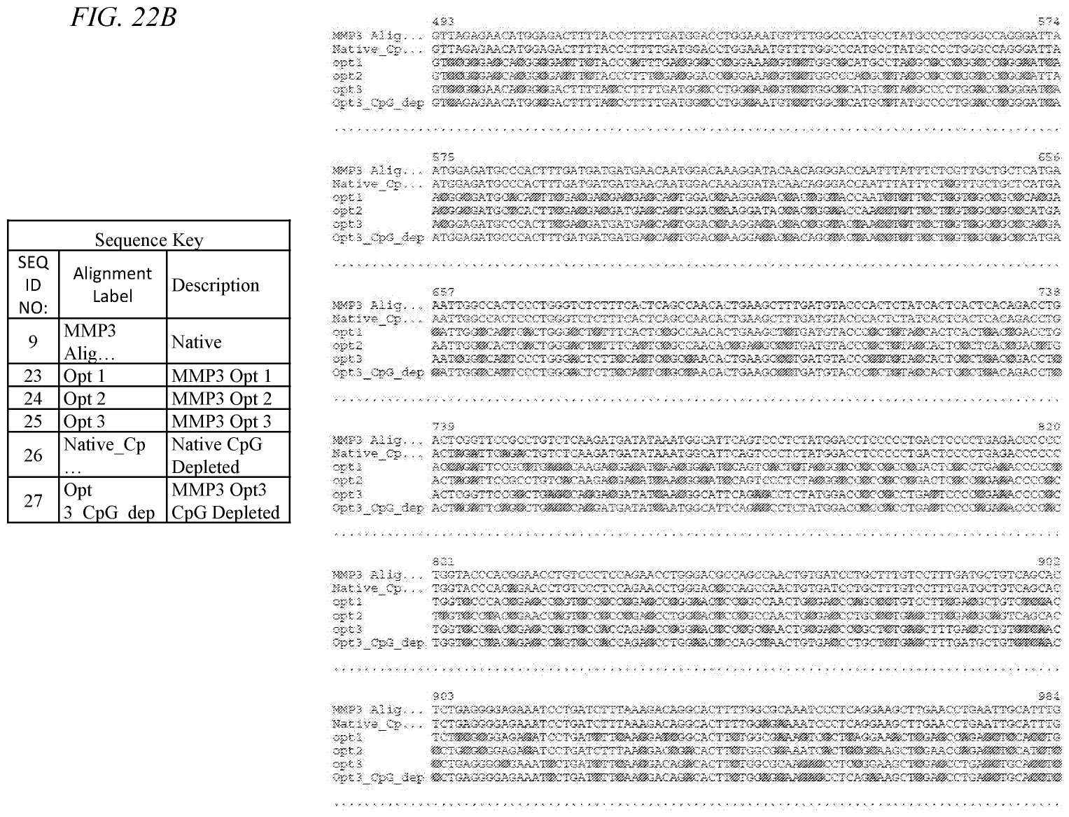

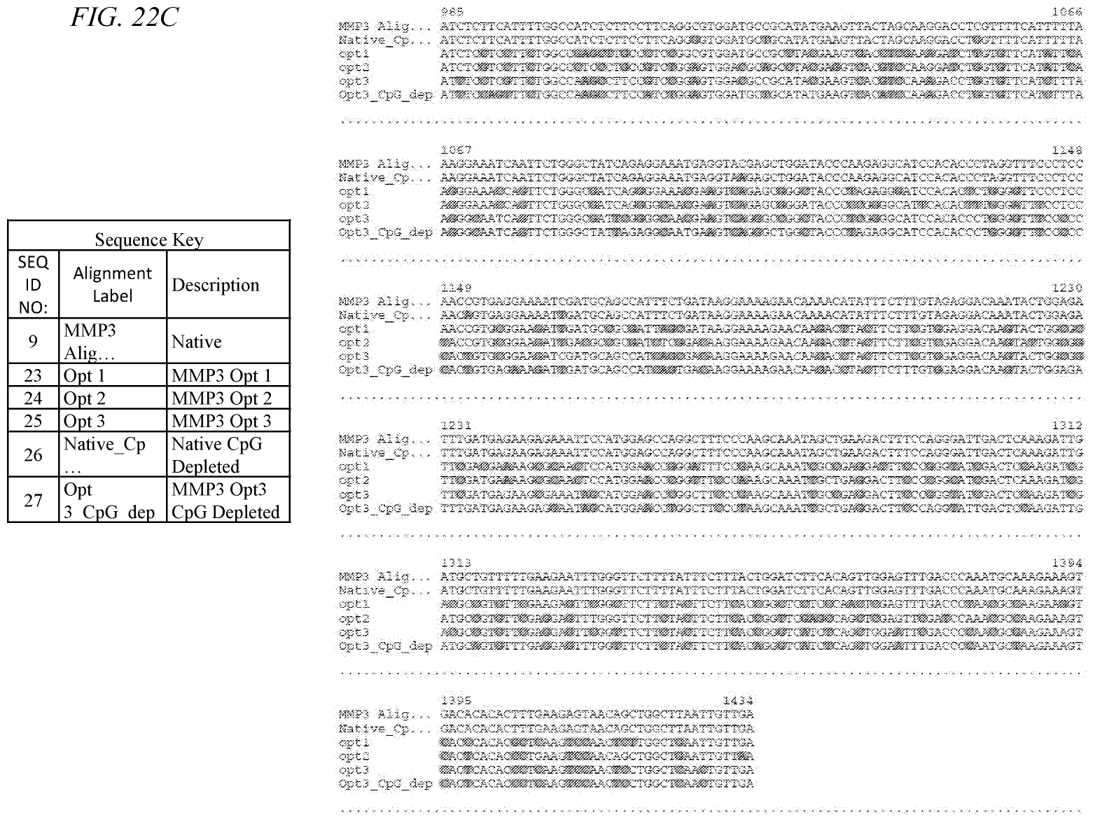

Codon optimization of the MMP3 sequence (SEQ ID NO: 9) was performed using algorithms to optimize the sequence for human codon usage. The native MMP3 sequence and one of the codon-optimized sequences, MMP Opt 3, were also modified for CpG depletion (Tables 5 and 6). Table 5 shows the sequence similarity of each optimized sequence to the native MMP3 sequence. Both the percent identity and the GC content of the codon-optimized sequences were significantly different than the native MMP3 sequence. FIGS. 22A-22C show alignments for the native and optimized sequences. Any of the optimized sequences are tested and characterized using the methods and analysis provided in the examples described herein.

TABLE 5
Comparison of codon-optimized MMP3
sequences to native sequence.

| Sequence | % Identity | % GC content |
|---|---|---|
| Native (SEQ ID NO: 9) | 100 | 45.89 |
| MMP3 Opt 1 (SEQ ID NO: 23) | 75.66 | 57.32 |
| MMP3 Opt 2 (SEQ ID NO: 24) | 75.80 | 57.25 |
| MMP3 Opt 3 (SEQ ID NO: 25) | 76.01 | 57.60 |
| Native CpG Depleted | 98.40 | 44.6 |
| (SEQ ID NO: 26) | | |
| MMP3 Opt3 CpG Depleted | 79.57 | 49.7 |
| (SEQ ID NO: 27) | | |

A pairwise comparison of the codon-optimized sequences in Table 6 shows the sequence similarities between the optimized sequences. The percent identities show the codon-optimized sequences have significant differences.

TABLE 6
Pairwise comparison of codon-optimized MMP3 sequences.

| | | | | % Identity | % Identity |
|---|---|---|---|---|---|
| | % Identity | % Identity | % Identity | Native | MMP3 Opt 3 |
| | MMP3 Opt 1 | MMP3 Opt 2 | MMP3 Opt 3 | CpG Depleted | CpG Depleted |
| | (SEQ ID | (SEQ ID | (SEQ ID | (SEQ ID | (SEQ ID |
| Sequence | NO: 23) | NO: 24) | NO: 25) | NO: 26) | NO: 27) |
| MMP3 Opt 1 | 100 | 84.73 | 83.82 | 75.31 | 79.78 |
| (SEQ ID | | | | | |
| NO: 23) | | | | | |
| MMP3 Opt 2 | 84.73 | 100 | 84.73 | 75.52 | 79.71 |
| (SEQ ID | | | | | |
| NO: 24) | | | | | |
| MMP3 Opt 3 | 83.82 | 82.91 | 100 | 75.52 | 90.66 |
| (SEQ ID | | | | | |
| NO: 25) | | | | | |
| Native CpG | 75.31 | 75.52 | 75.52 | 100 | 80.75 |
| Depleted | | | | | |
| (SEQ ID | | | | | |
| NO: 26) | | | | | |
| MMP3 Opt3 | 79.98 | 79.71 | 90.66 | 80.75 | 100 |
| CpG Depleted | | | | | |
| (SEQ ID | | | | | |
| NO: 27) | | | | | |

### Expression of Codon-Optimized Sequences In Vitro

Three of the optimized sequences were chosen for characterization, MMP3 Opt 1 (SEQ ID NO: 23), MMP3 Opt 2 (SEQ ID NO: 24), and MMP3 Opt 3 (SEQ ID NO: 25). The codon-optimized sequences were produced and sub-cloned into an AAV expression cassette. Plasmids containing codon-optimized sequences were transfected into HEK293 cells, representing a human cell line easily transfectable and widely used, and HCEC (human corneal endothelial cells), representing the intended target cell type. In both cases, cells were transfected for 48 hours using the lipofectamine 3000 transfection reagent. Protein was then taken from the culture media supernatant, and also from the cell lysates. ELISA for human MMP3 (R&D systems, DMP300) was performed to determine the MMP3 concentration of each sample. A BCA assay was performed to determine total protein concentration. ELISA data was normalized to the BCA data to generate the amount of MMP3 in ng per μg of total protein.

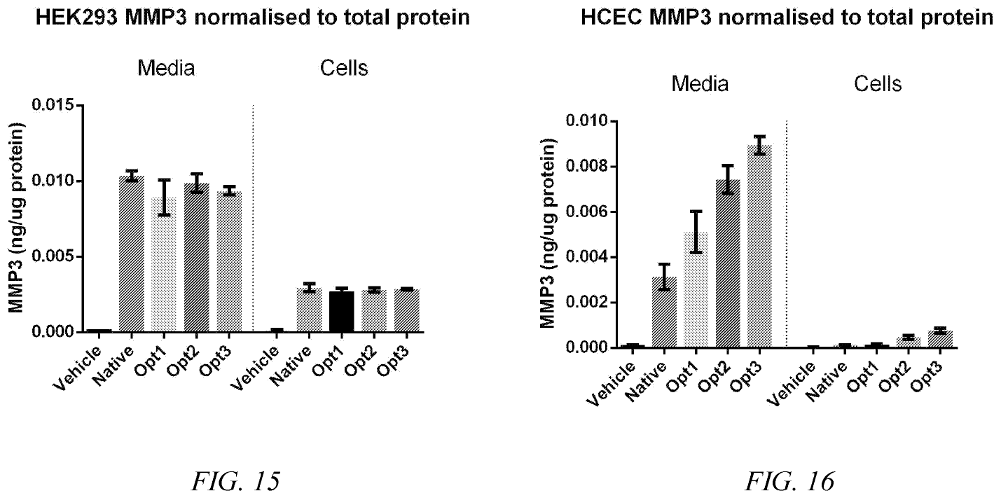

Expression of codon-optmizied MMP3 sequences was characterized in HEK and HCEC cell lines. The optimized constructs transfected into HEK cells showed little change in MMP3 expression in both the media and cell lysates for this cell type (FIG. 15). In contrast, MMP3 expression in HCEC cells showed a clear difference in the expression between the different sequences encoding MMP3 (FIG. 16). Analysis of optimized constructs transfected into HCEC cells shows significant increases in MMP3 protein production in both the cell lysate and the media supernatant compared to the native MMP3 encoding sequence. Differences in protein production between the optimized sequences show that the MMP3 Opt 3 sequence resulted the highest level of protein production, followed by MMP3 Opt 2 and MMP3 Opt 1 (FIG. 16). These results suggest that in the relevant cell type, the codon optimized sequences result in greater MMP3 protein production compared to the native sequence.

Comparison of expression trends observed for the native and codon optimized sequences encoding MMP3 in HEK293 and HCEC cells show that expression cannot be predicted between human cell lines. In HEK293 cells there was very little variability in expression between the native and codon optimized sequences, and between the codon-optimized sequences. Unexpectedly, every codon-optimized sequence was expressed at a higher level than the native sequence and there was differential expression between the codon-optimized sequences in HCEC cells. This results demonstrates improved expression is not an obvious consequence of codon-optimization of an MMP3 encoding sequence.

### AAV9 Viral Vector Delivery of Codon-Optimized MMP3 Sequences

AAV9 viral vectors were produced containing either the most efficient codon optimized sequence, MMP3 Opt 3, or the native MMP3 sequence. The vectors were transduced into HCEC cells at an MOI (multiplicity of infection) of 1×10⁵. Media supernatant was harvested 48 hours post-transfection. ELISA, western blot and FRET (fluorescent resonance energy transfer) activity assay were performed on these samples to characterize protein expression and protease activity.

### Expression of Codon-Optimized MMP3 Sequence Delivered by AAV9 Viral Vector

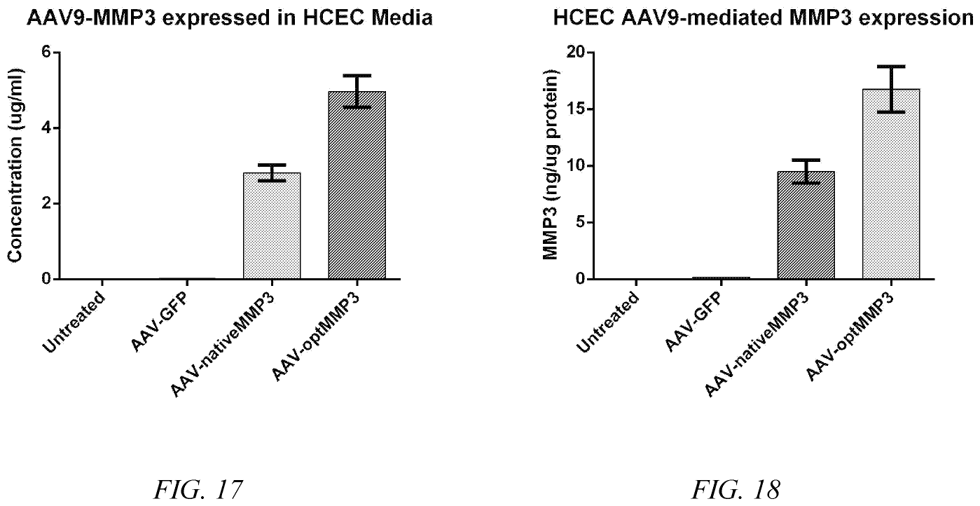

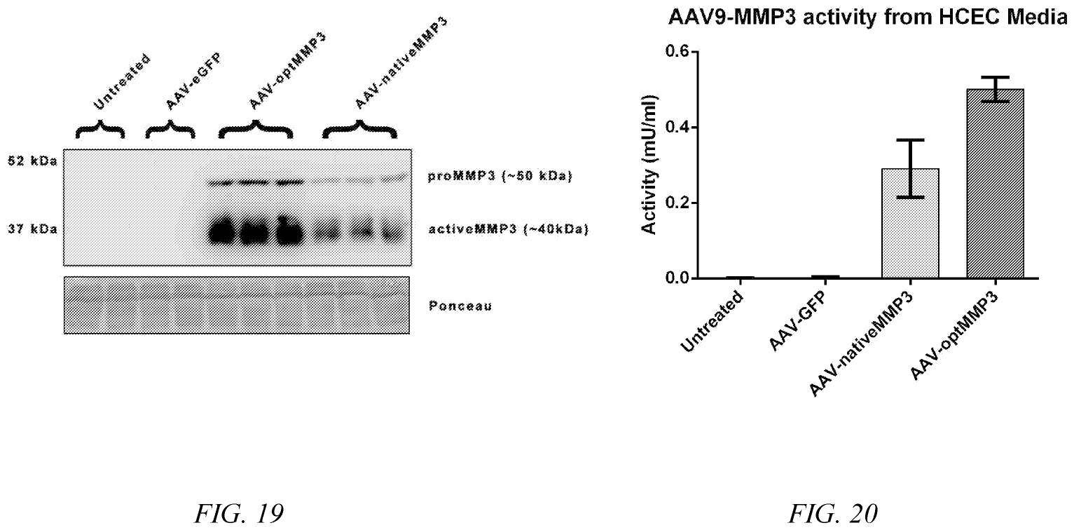

MMP3 expression was assessed in HCEC cells transduced with the AAV9 vectors. MMP3 expression was significantly increased in the media when using the MMP3 Opt 3 construct compared to controls and the construct containing the native MMP3 sequence (P=<0.0001, N=16 MMP3 Opt 3, N=15 native, unpaired t-test) (FIG. 17). Further, the MMP3 levels were further normalized normalized to total protein concentration where available. (P=<0.0001, N=8) (FIG. 18). Western blot analysis was used to measure MMP3 secreted into media from codon-optimized sequences and native sequences in HCEC cells transduced by the AAV9 vectors. Media from HCEC cell cultures treated with AAV9 expressing native or optimized MMP3 were immunoblotted for MMP3 (Abcam, #ab52915) (FIG. 19). The results show stronger intensity of both pro-MMP3 and active MMP3 bands in lanes containing optimized construct-treated cells. Ponceau is presented as a loading control.

### Protease Activity of Codon-Optimized MMP3 Sequence Delivered by AAV9 Viral Vector

MMP3 protease activity in media harvested from cells transduced by the AA9 vecotrs. (FIG. 20). Protease activity of the expressed MMP3 was assessed using an MMP3 activity assay FRET kit (Abcam, #ab118972). MMP3 activity was significantly increased using the optimized construct. (P<0.0001, N =8). Activity was quantified in mU/ml, where one unit is defined as the amount of enzyme that will generate 1.0 μmol of unquenched Mca per minute at room temperature, according to the manufacturers protocol.

### Example 7: Effect of MMP3 on Outflow Facility in Human Eyes

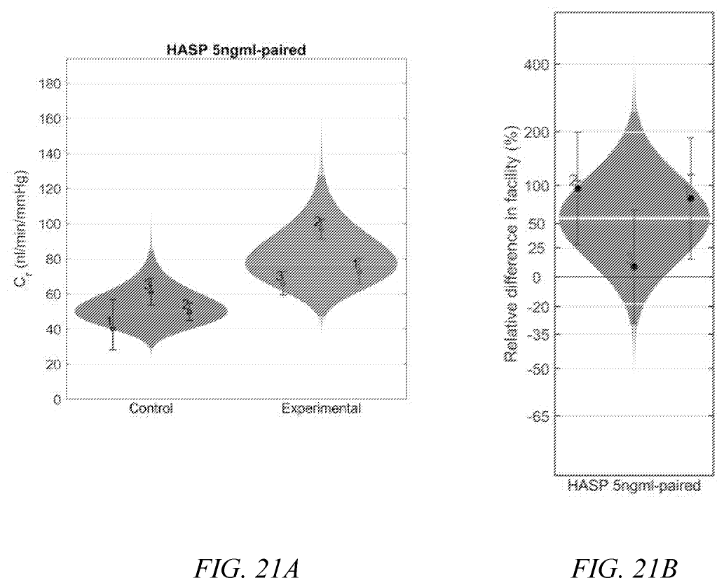

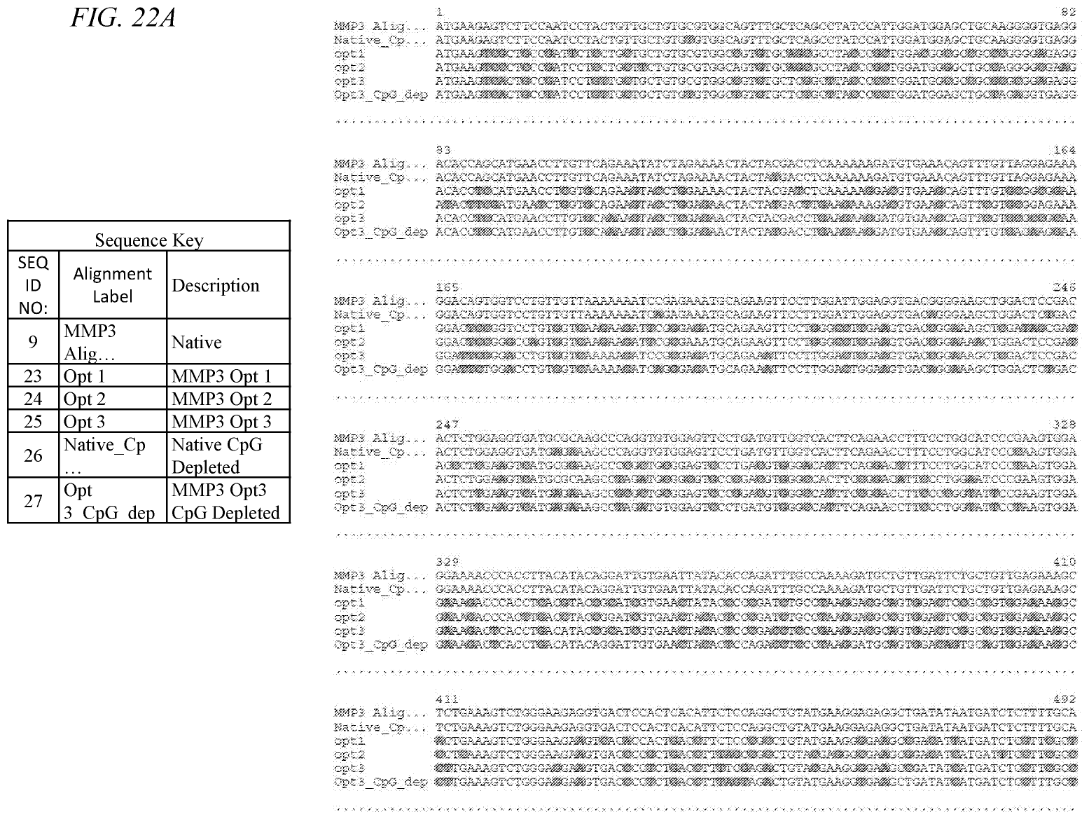

To expand on mouse and non-human primate data, the efficacy of recombinant human MMP3 on human eyes was determined. Post-mortem eyes were bisected, and the anterior chamber mounted to an iPerfusion system. MMP3 was perfused at incrementing concentrations over several days into the eyes after stable flow rates were achieved. A vehicle was used in the contralateral eye, allowing for paired observations. The 5 ng/ml concentration was chosen for analysis. At this concentration, MMP3 increased outflow facility on average by 56% when compared to controls, one hour after a 5 ng/ml perfusate was introduced to the anterior chamber via exchange, P=0.1399, N=3 pairs (FIG. 21A-21B). These results suggest exposure to recombinant MMP3 in the eyes of glaucoma patients can increase outflow facility and reduce intraocular pressure, particularly when a dose of recombinant protein or gene therapy vector is selected to achieve a dose of 1-10 ng/ml.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

The section headings used herein are for organizational purposes only and are not to be construed as limiting the subject matter described.

While illustrative embodiments have been illustrated and described, it will be appreciated that various changes can be made therein without departing from the spirit and scope of the invention.

What is claimed:

1. A unit dose comprising a plurality of recombinant adeno-associated virus of serotype 9 (rAAV9) particles,
wherein each rAAV9 of the plurality of rAAV9 particles is non-replicating, and
wherein each rAAV9 of the plurality of rAAV9 particles comprises a polynucleotide comprising, from 5′ to 3′:
(a) a sequence encoding a 5′ inverted terminal repeat (ITR);
(b) a sequence encoding a promoter;
(c) a sequence encoding a human matrix metalloproteinase 3 (hMMP-3);
(d) a sequence encoding a polyadenylation (polyA) signal; and
(e) a sequence encoding a 3′ ITR; and
wherein the unit dose comprises between 1×10¹⁰ vector genomes (vg) and 5×10¹² vg, inclusive of the endpoints, of rAAV9 particles.

2. The unit dose of claim 1, wherein the unit dose is (i) sterile and (ii) comprises a pharmaceutically acceptable carrier.

3. The unit dose of claim 1 or claim 2, wherein each rAAV9 of the plurality of rAAV9 particles is a single-stranded AAV (ssAAV) vector.

4. The unit dose of claim 1 or claim 2, wherein each rAAV9 of the plurality of rAAV9 particles is a self-complementary AAV (scAAV) vector.

5. The unit dose of any one of claims 1-4, wherein the promoter comprises a CMV promoter, and wherein the sequence encoding the CMV promoter comprises or consists of the sequence of SEQ ID NO: 6, SEQ ID NO: 7, or SEQ ID NO: 19, or a functional variant thereof, optionally having 80%, 90%, 95%, or 99% sequence identity thereto.

6. The unit dose of any one of claims 1-5, wherein the sequence encoding human MMP-3 comprises or consists of a nucleotide sequence encoding the MMP-3 amino acid sequence of SEQ ID NO: 1, SEQ ID NO: 2, SEQ ID NO: 10 or SEQ ID NO: 22, or a functional variant thereof, optionally having 80%, 90%, 95%, or 99% sequence identity thereto.

7. The unit dose of any one of claims 1-5, wherein the nucleotide sequence encoding the MMP-3 amino acid sequence comprises a wild-type nucleotide sequence.

8. The unit dose of claim 7, wherein the sequence encoding MMP-3 comprises or consists of the nucleotide sequence of SEQ ID NO: 9, SEQ ID NO: 23, SEQ ID NO: 24, SEQ ID NO: 25, SEQ ID NO: 26, or SEQ ID NO: 27, or shares at least 80%, 90%, 95%, 97%, 99% sequence identity to thereto.

9. The unit dose of any one of claims 1-8, wherein the sequence encoding the 5′ ITR is derived from a 5′ ITR sequence of an AAV of serotype 2 (AAV2).

10. The unit dose of any one of claims 1-9, wherein the sequence encoding the 5′ ITR comprises a sequence that is identical to a sequence of a 5′ ITR of an AAV2.

11. The unit dose of any one of claims 1-10, wherein the sequence encoding the 5′ ITR comprises or consists of the nucleotide sequence of SEQ ID NO: 5, SEQ ID NO: 14, or SEQ ID NO: 15.

12. The unit dose of any one of claims 1-11, wherein the sequence encoding the 3′ ITR is derived from a 3′ ITR sequence of an AAV2.

13. The unit dose of any one of claims 1-12, wherein the sequence encoding the 3′ ITR comprises a sequence that is identical to a sequence of a 3′ ITR of an AAV2.

14. The unit dose of any one of claims 1-13, wherein the sequence encoding the 3′ ITR comprises or consists of the nucleotide sequence of SEQ ID NO: 12 or any one of SEQ ID NOs: 16-18.

15. The unit dose of any one of claims 1-14, wherein the sequence encoding the polyA signal comprises a human growth hormone (hGH) polyA sequence.

16. The unit dose of claim 15, wherein the sequence encoding the hGH polyA signal comprises the nucleotide sequence of SEQ ID NO: 11.

17. The unit dose of any one of claims 1-16, wherein the polynucleotide further comprises a Kozak sequence.

18. The unit dose of claim 17, wherein the Kozak sequence comprises or consists of the nucleotide sequence of CGCCACCATG (SEQ ID NO: 21).

19. The unit dose of claim of any one of claims 1-18, wherein the polynucleotide comprises or consists of the sequence of SEQ ID NO: 3 or SEQ ID NO: 4.

20. The unit dose of any one of claims 1-19, wherein each of the rAAV9 particles comprise a viral Cap protein isolated or derived from an AAV serotype 9 (AAV9) Cap protein.

21. A unit dose comprising recombinant matrix metalloproteinase 3 (MMP-3) protein, wherein the unit dose comprises between 1 milligrams per milliliter (mg/mL) and 500 mg/mL, inclusive of the endpoints, of the recombinant MMP-3 protein; or between 0.1 nanograms (ng) and 10 ng, inclusive of the endpoints, of the recombinant MMP-3 protein.

22. The unit dose of claim 21, wherein the unit dose comprises about 0.01 to about 10 ng/mL of the recombinant MMP-3 protein.

23. The unit doses of claim 21 or claim 22, wherein the recombinant MMP-3 protein is a human MMP-3 protein.

24. The unit dose of any one of claims 21-23, wherein the recombinant MMP-3 protein has a polypeptide sequence that comprises or consist of the sequence of SEQ ID NO: 1, SEQ ID NO: 2, SEQ ID NO: 10 or SEQ ID NO: 22, or a functional variant or functional fragment thereof, optionally having 80%, 90%, 95%, or 99% sequence identity thereto.

25. A method of transducing the corneal endothelium of a subject, comprising administering an effective amount of the unit dose of any one of claims 1-24,
wherein the subject is a primate.

26. The method of claim 25, wherein each rAAV9 of the plurality of rAAV9 particles in the unit dose is a single-stranded AAV (ssAAV).

27. The method of claim 25 or claim 26, wherein administering the effective amount of the unit dose results in expression of MMP-3 in the aqueous humor of an eye of the subject at a measured concentration of between 0.01 ng/mL and about 10 ng/mL, inclusive of the endpoints, between 0.01 ng/mL and about 500 ng/mL, inclusive of the endpoints, or between 0.01 ng/mL and about 1000 ng/mL, inclusive of the endpoints.

28. The method of any one of claims 25-27, wherein the measured concentration is greater than or equal to 1 ng/mL.

29. The method of any one of claims 25-28, wherein the measured concentration is less than or equal to 10 ng/mL.

30. The method of any one of claims 25-29, wherein the measured concentration is 1-10 ng/mL, inclusive of the endpoints.

31. The method of claim 28, wherein the measured concentration is at least 1-3 ng/mL, inclusive of the endpoints.

32. The method of any one of claims 25-31, wherein the expression of MMP-3 is maintained at least 21 days, 42 days, 56 days, or 66 days.

33. The method of any one of claims 25-31, wherein the expression of MMP-3 is maintained at least 90 days.

34. The method of any one of claims 25-31, wherein the expression of MMP-3 in aqueous humor is measured by Western Blot or ELISA.

35. The method of any one of claims 25-34, wherein the method increases outflow facility by at least 25% or by at least 30%.

36. The method of claim 35, wherein the increase in outflow facility occurs within about 66 days of the administering step.

37. The method of any one of claims 25-36, wherein the corneal thickness remains unchanged relative to corneal thickness in the subject before the administering step and/or relative to corneal thickness in a subject administered a control unit dose.

38. The method of any one of claims 25-37, wherein the method causes no inflammatory response.

39. The method of any one of claims 25-38, wherein the method results in serum levels of MMP-3 that are not elevated over a baseline level of MMP-3 in the serum of the subject.

40. The method of any one of claims 25-39, wherein the administering step comprises intracameral injection of the unit dose into at least one eye of the subject.

41. A method of reducing intraocular pressure (IOP) in at least one eye of a subject, comprising administering an effective amount of the unit dose of any one of claims 1-24
wherein the subject is a primate.

42. The method of claim 41, wherein administering the effective amount of the unit dose results in expression of MMP-3 in the aqueous humor of an eye of the subject at a measured concentration of between 0.01 ng/mL and about 10 ng/mL, inclusive of the endpoints.

43. The method of claim 42, wherein the measured concentration is greater than or equal to 1 ng/mL.

44. The method of claim 42 or claim 43, wherein the measured concentration is less than or equal to 10 ng/mL.

45. The method of any one of claims 42-44, wherein the measured concentration is 1-10 ng/mL, inclusive of the endpoints.

46. The method of any one of claims 42-44, wherein the measured concentration is at least 1-3 ng/mL, inclusive of the endpoints.

47. The method of any one of claims 42-46, wherein the expression of MMP-3 is maintained at least 21 days, 42 days, 56 days, or 66 days.

48. The method of any one of claims 42-47, wherein the expression of MMP-3 is maintained at least 90 days.

49. The method of any one of claims 42-48, wherein the expression of MMP-3 is measured by Western Blot or ELISA.

50. The method of any one of claims 42-49, wherein the method increases outflow facility by at least 25% or by at least 30%.

51. The method of any one of claims 42-50, wherein the method reduces intraocular pressure (IOP).

52. The method of any one of claims 42-51, wherein the corneal thickness remains unchanged relative to corneal thickness in the subject before the administering step and/or relative to corneal thickness in a subject administered a control unit dose.

53. The method of any one of claims 42-52, wherein the method causes no inflammatory response.

54. The method of any one of claims 42-53, wherein the method results in serum levels of MMP-3 that are not elevated over a baseline level of MMP-3 in the serum of the subject.

55. The method of any one of claims 42-54, wherein the administering step comprises injection of the unit dose into the cornea of at least one eye of the subject.

56. The method of any one of claims 42-55, wherein the administering step comprises injection of the unit dose into the temporal cornea of at least one eye of the subject.

57. The method of any one of claims 42-56, wherein the administering step comprises intracameral injection of the unit dose into at least one eye of the subject.

58. A method of treating and/or preventing elevated IOP and/or glaucoma in a subject in need thereof, comprising administering an effective amount of the unit dose of any one of claims 1-24 to the subject,
wherein the subject is a primate.

59. A method of transducing the corneal endothelium of a subject, comprising administering an effective amount of a unit dose comprising a plurality of recombinant adeno-associated virus of serotype 9 (rAAV9) particles to the subject,
wherein the subject is a primate;
wherein each rAAV9 of the plurality of rAAV9 particles is non-replicating;
wherein each rAAV9 of the plurality of rAAV9 particles is a single-stranded AAV (ssAAV);
wherein each rAAV9 of the plurality of rAAV9 particles comprises a polynucleotide comprising, from 5′ to 3′:
(a) a sequence encoding a 5′ inverted terminal repeat (ITR);
(b) a sequence encoding a promoter;
(c) a sequence encoding a matrix metalloproteinase 3 (MMP-3);
(d) a sequence encoding a polyadenylation (polyA) signal; and
(e) a sequence encoding a 3′ ITR; and
wherein the unit dose comprises
(i) between 1×10¹⁰ vector genomes (vg) and 5×10¹² vg, inclusive of the endpoints, of rAAV9 particles; or
(ii) about 1×10¹¹ vector genomes (vg) per milliliter (mL) to 1×10¹⁴ vg/mL of rAAV9 particles; and
wherein administering the effective amount of the unit dose results in expression of MMP-3 in the aqueous humor of an eye of the subject at a measured concentration of between 0.01 ng/mL and about 10 ng/mL, inclusive of the endpoints.

60. The method of claim 59, wherein the sequence encoding MMP-3 comprises or consists of the nucleotide sequence of SEQ ID NO: 9, SEQ ID NO: 23, SEQ ID NO: 24, SEQ ID NO: 25, SEQ ID NO: 26, or SEQ ID NO: 27, or shares at least 80%, 90%, 95%, 97%, 99% sequence identity to thereto.

61. A method of transducing the corneal endothelium of a subject, comprising administering an effective amount of a unit dose comprising a plurality of recombinant adeno-associated virus of serotype 9 (rAAV9) particles to the subject,
wherein the subject is a primate;
wherein each rAAV9 of the plurality of rAAV9 particles is non-replicating;
wherein each rAAV9 of the plurality of rAAV9 particles is a single-stranded AAV (ssAAV);
wherein each rAAV9 of the plurality of rAAV9 particles comprises a polynucleotide comprising, from 5′ to 3′:
(a) a sequence encoding a 5′ inverted terminal repeat (ITR);
(b) a sequence encoding a promoter;
(c) a sequence encoding a transgene;
(d) a sequence encoding a polyadenylation (polyA) signal; a
(e) a sequence encoding a 3′ ITR.

62. A gene therapy vector comprising an expression cassette comprising a transgene encoding a human matrix metalloproteinase 3 (hMMP-3) or a functional variant thereof, optionally operatively linked to a promoter, wherein the transgene is optimized for expression in a human host cell.

63. The gene therapy vector of claim 62, wherein the human host cell is a human corneal endothelial cell.

64. The gene therapy vector of claim 62 or claim 63, wherein the transgene shares at least 80% identity, at least 85% identity, at least 90% identity, at least 95% identity, at least 97% identity, or at least 99% identity to a sequence selected from SEQ ID NOs: 23-27.

65. The gene therapy vector of claim 64, wherein the transgene comprises a sequence selected from SEQ ID NOs: 23-27.

66. The gene therapy vector of claim 65, wherein the transgene shares at least 95% identity to SEQ ID NO: 23 or is identical to SEQ ID NO: 23.

67. The gene therapy vector of claim 65, wherein the transgene shares at least 95% identity to SEQ ID NO: 24 or is identical to SEQ ID NO: 24.

68. The gene therapy vector of claim 65, wherein the transgene shares at least 95% identity to SEQ ID NO: 25 or is identical to SEQ ID NO: 25.

69. The gene therapy vector of claim 65, wherein the transgene shares at least 95% identity to SEQ ID NO: 26 or is identical to SEQ ID NO: 26.

70. The gene therapy vector of claim 65, wherein the transgene shares at least 95% identity to SEQ ID NO: 27 or is identical to SEQ ID NO: 27.

71. The gene therapy vector of any one of claims 63-70, wherein the vector is an adeno-associated virus (AAV) vector.

72. The gene therapy vector of claim 71, wherein the AAV vector is an AAV9 vector.

73. The gene therapy vector of claim 71 or claim 72, wherein the AAV vector is a single-stranded AAV (ssAAV) vector.

74. The gene therapy vector of claim 71 or claim 72, wherein the AAV vector is a self-complementary AAV (ssAAV) vector.

75. A pharmaceutical composition comprising the gene therapy vector of any one of claims 62 to 74.

76. A method of treating and/or preventing elevated IOP and/or glaucoma in a subject in need thereof, comprising administering an effective amount of the gene therapy vector of any one of claims 62-74 or the pharmaceutical composition of claim 75 to the subject,
wherein the subject is a primate.

77. A polynucleotide, comprising a transgene encoding a human matrix metalloproteinase 3 (hMMP-3) or a functional variant thereof, wherein the transgene is optimized for expression in a human host cell.

78. The polynucleotide of claim 77, wherein the polynucleotide comprises a promoter operatively linked to the transgene.

79. The polynucleotide of claim 77 or claim 78, wherein the human host cell is a human corneal endothelial cell.

80. The polynucleotide of any one of claims 77-79, wherein the transgene shares at least 80% identity, at least 85% identity, at least 90% identity, at least 95% identity, at least 97% identity, or at least 99% identity to a sequence selected from SEQ ID NOs: 23-27.

81. The polynucleotide of claim 80, wherein the transgene comprises a sequence selected from SEQ ID NOs: 23-27.

82. The polynucleotide of claim 81, wherein the transgene shares at least 95% identity to SEQ ID NO: 23 or is identical to SEQ ID NO: 23.

83. The polynucleotide of claim 81, wherein the transgene shares at least 95% identity to SEQ ID NO: 24 or is identical to SEQ ID NO: 24.

84. The polynucleotide of claim 81, wherein the transgene shares at least 95% identity to SEQ ID NO: 25 or is identical to SEQ ID NO: 25.

85. The polynucleotide of claim 81, wherein the transgene shares at least 95% identity to SEQ ID NO: 26 or is identical to SEQ ID NO: 26.

86. The polynucleotide of claim 81, wherein the transgene shares at least 95% identity to SEQ ID NO: 27 or is identical to SEQ ID NO: 27.

87. The polynucleotide of any one of claims 77-86, wherein the polynucleotide comprises adeno-associated virus (AAV) terminal repeats (ITRs) flanking the transgene.

88. The polynucleotide of any one of claims 77-87, wherein the polynucleotide is an isolated polynucleotide.

89. An isolated cell, comprising the polynucleotide of any one of claims 77-88.

90. A pharmaceutical composition, comprising the polynucleotide of any one of claims 77-88.
